(19) United States
(12) Patent Application Publication
(10) Pub. No.: US 2025/0295421 A1
(43) Pub. Date: Sep. 25, 2025

(54) EXTENSIBLE CATHETER AND METHOD FOR REMOVING AN OCCLUSION FROM A PATIENT'S VASCULATURE

(71) Applicant: Jeffrey E. THOMAS, Hillsborough, CA (US)

(72) Inventor: Jeffrey E. THOMAS, Hillsborough, CA (US)

(21) Appl. No.: 19/036,785

(22) Filed: Jan. 24, 2025

Related U.S. Application Data
(60) Provisional application No. 63/624,636, filed on Jan. 24, 2024.
(60) Provisional application No. 63/640,112, filed on Apr. 29, 2024.

(51) Int. Cl. A61B 17/22; A61B 17/00; A61M 25/00

(52) U.S. Cl. CPC A61B 17/22; A61M 25/0021; A61M 25/007; A61B 2017/00557; A61B 2017/00778; A61B 2017/22038; A61B 2017/22067; A61B 2017/22079; A61B 2217/005; A61M 2025/0004; A61M 2025/0042

(57) ABSTRACT

A microcatheter system for removing an occlusion from a patient's vasculature, comprising an outer tubular member having an outer surface, a proximal end, a distal end, and a central lumen extending from the proximal end toward the distal end, wherein the central lumen and the outer surface cooperate to define a side wall radially therebetween, one or more perforations formed in the side wall of the outer tubular member near the distal end thereof, the one or more perforations being in fluid communication with the central lumen of the outer tubular member, an inner tubular member slidably positioned within the central lumen of the outer tubular member, the inner tubular member having a distal end extending distally from a distal opening in the outer tubular member; and, an extensible member having a proximal end affixed to the outer tubular member and a distal end affixed to the inner tubular member, wherein the inner tubular member is advanceable distally relative to the outer tubular member to expand the extensible member.

### CROSS-REFERENCE TO RELATED APPLICATIONS

The present application claims priority under 35 US 119 to U.S. provisional patent application Ser. No. 63/624,636, filed on Jan. 24, 2024, currently pending, and to U.S. provisional patent application Ser. No. 63/640,112, filed on Apr. 29, 2024, currently pending, the written descriptions of which are both hereby incorporated by reference as if fully rewritten herein.

### BACKGROUND

The present disclosure relates generally to apparatuses and methods for treating and removing an occlusion from within a patient's vasculature, including the arteries of the brain, the arteries surrounding the heart, and any branch artery stemming therefrom. More particularly, the present disclosure relates to apparatuses, systems and methods for removing an occlusion from within a small artery of a patient's vasculature, in which an expandable microcatheter is used to physically capture and extract the occlusion therefrom.

Thrombotic or embolic occlusion of a cerebral artery is often the cause of an ischemic or acute stroke event. It is often characterized by a sudden loss of specific neurologic function, or even death, due to loss of circulation of blood caused by the occlusion to a specific area of the brain “downstream” from the occlusion. For instance, an occlusion in the middle cerebral artery (MCA) or its branches is the most common type of anterior circulation infarct, accounting for about two-thirds of all first strokes. Immediately following such an acute stroke event, preferably within eight hours from the initial onset of the occlusion, it often is necessary to re-establish blood flow through the occluded MCA in order to supply fresh blood to the specific area of the brain that had been deprived of blood while the occlusion was present. If the occlusion is not resolved quickly, the ischemia may lead to permanent neurologic deficit or even death.

Conventional methods for resolving an occlusion involve advancing a microcatheter through the patient's vasculature via percutaneous access made, for instance, in the patient's radial, ulnar or femoral artery, and using conventional guidewires, steering catheters, etc., in order to “steer” a distal tip of the microcatheter through the patient's vasculature toward the occlusion. Once the microcatheter is advanced through the patient's vasculature such that the distal tip of the microcatheter is positioned proximate the occlusion, the microcatheter is used to resolve the occlusion. For instance, it is known to utilize a lumen provided in the microcatheter to deliver treatment solutions such as arterialized or oxygenated blood, thrombolytic agents, cold plasma, saline etc., through an opening in the distal tip of the microcatheter to the site of the occlusion, which such treatment solutions can be used to dissolve or disintegrate the occlusion, thereby re-establishing blood flow through the affected artery and restoring blood supply to the specific area of the brain that had been deprived of blood while the occlusion was present.

Not all treatment solutions, however, are effective to completely resolve an occlusion. For instance, thrombolytic agents such as tPA are not always effective to treat a so-called “white thrombus” (i.e. fibrin/platelet thrombus) as tPA has no activity against this type of occlusion. As such, using thrombolytic agents, alone, may not be sufficient to fully resolve all occlusions. And in any event, utilizing such treatment solutions sometimes results in the occlusion breaking up into numerous loose fragments which cannot be permitted to traverse the patient's vasculature freely to thereby create a risk of pulmonary embolism, etc. As such, there is a need to provide an apparatus, system and method for treating a vascular occlusion that more fully and completely removes the vascular occlusion than can be achieved using conventional treatment solutions alone.

For example, it is known to adapt the distal end of the microcatheter, such as by providing perforations in a sidewall thereof, to suction out the fragments of the occlusion that are created as a result of applying the treatment solution thereto. It also is known to use the suction, such as by vacuum or negative pressure system, to physically adhere larger fragments of the occlusion to the sidewall of the microcatheter as the microcatheter is being removed from the patient's vasculature, thereby physically removing the fragments of the occlusion therewith. U.S. Pat. No. 10,932,797 and US Patent Publication No. 2021/0219998, the complete disclosures of which are hereby incorporated herein by reference as if fully rewritten herein, are two examples of devices for delivering a treatment solution to an occlusion in a patient's vasculature via a microcatheter and for recovering fragments of the occlusion created thereby utilizing a source of suction, such as a vacuum or negative pressure system. However, there is a need to provide an apparatus, system and method for treating a vascular occlusion that even more fully and completely removes the vascular occlusion than can be achieved using a source of suction alone.

Despite the advances made using conventional apparatuses, systems and methods, there is a need for additional ways to ensure the occlusion is more fully and completely removed from the patient's vasculature.

### SUMMARY OF THE DISCLOSURE

Embodiments of the present invention provide apparatuses, systems and methods to physically capture an occlusion present within a patient's vasculature in which a microcatheter may be utilized along with a source of suction to ensure that the occlusion, or fragments thereof, stays adhered to the microcatheter, or to the side walls thereof, while the microcatheter is removed from the patient's vasculature. In further embodiments of the present invention, apparatuses, systems and methods are provided to physically capture an occlusion, or fragments thereof, present in a patient's vasculature in which perforations may be provided in a sidewall of a microcatheter near a distal end thereof to physically capture, grasp, grip, hold or retain the occlusion, or the fragments thereof, against the sidewall of the microcatheter as the microcatheter is removed from the patient's vasculature, thereby removing the occlusion therewith. In even further embodiments of the present invention, apparatuses, systems and methods are provided to physically capture an occlusion, or fragments thereof, present in a patient's vasculature in which an extensible microcatheter may be utilized along with perforations provided in a sidewall thereof near a distal end thereof to “open” and “close” such perforations, thereby enhancing the perforations' ability to capture grasp, grip, hold or retain the occlusion, or the fragments thereof, against the sidewall of the microcatheter as the microcatheter is removed from the patient's vasculature, thereby more fully and completely removing the occlusion therewith. In yet even further embodiments, a source of suction may be provided, such as by vacuum or negative pressure system, in communication with the perforations to even further enhance the perforations' ability to capture grasp, grip, hold or retain the occlusion, or the fragments thereof, against the sidewall of the microcatheter as the microcatheter is removed from the patient's vasculature, thereby more fully and completely removing the occlusion therewith.

According to an embodiment of the invention, it is advantageous to construct the extensible microcatheter using a flexible, expandable or “shape memory” material in order to allow the microcatheter to controllably move between a “relaxed” state and an “extended” state. Perforations provided in a sidewall of the microcatheter are thereby controllably moved between “relaxed” and “open” configurations as the microcatheter is moved between the “relaxed” and “extended” states, respectively, thereby further enhancing the microcatheter's ability to physically capture, grasp, grip, hold or retain the occlusion, or the fragments thereof, by the microcatheter as the microcatheter is removed from the patient's vasculature (removing the occlusion therewith). According to other embodiments of the invention, it is even more advantageous to utilize a source of suction, such as by vacuum or negative pressure system, in communication with the perforations to even further enhance the perforations' ability to capture grasp, grip, hold or retain the occlusion, or the fragments thereof, by the microcatheter as the microcatheter is removed from the patient's vasculature (removing the occlusion therewith).

### BRIEF DESCRIPTION OF THE FIGURES

The accompanying drawings, which are incorporated in and constitute a part of the specification, illustrate various example apparatuses, systems, methods, and so on, that illustrate various example embodiments of aspects of the invention. It will be appreciated that the illustrated element boundaries (e.g., boxes, groups of boxes, or other shapes) shown in the Figures represent one example of the boundaries. One of ordinary skill in the art will appreciate that one element may be designed as multiple elements or that multiple elements may be designed as one element. An element shown as an internal component of another element may be implemented as an external component and vice versa.

Exemplary embodiments of the present invention shown in the Figures may not necessarily be drawn to scale, with emphasis instead being placed on illustrating the various aspects and features of the illustrated embodiments. In addition, one or more hidden elements may be removed from certain Figures in order to enhance the clarity and understanding of the remaining elements of the various embodiments of the present invention shown in the Figures. Common reference numerals are used among the Figures to depict features or elements that are common to the various embodiments of the present invention.

FIG. 1 is a perspective view of an apparatus for removing an occlusion from a patient's vasculature according to an embodiment of the present invention in which a microcatheter is shown having an open distal end;

FIG. 2 is a perspective view of an apparatus for removing an occlusion from a patient's vasculature according to an embodiment of the present invention in which a microcatheter is shown having a closed distal end and wherein the microcatheter includes perforations in a sidewall thereof located proximal of the closed distal end;

FIG. 3 is a close-up perspective view of a distal end of an apparatus for removing an occlusion from a patient's vasculature according to an embodiment of the present invention in which a microcatheter is shown having perforations in a sidewall thereof located near the distal end between two inflatable members spaced along an axis of the microcatheter;

FIG. 4 is a side view of the distal end of the apparatus shown in FIG. 3, in which the distal end of the microcatheter is shown positioned within an artery of a patient's vasculature proximal of an occlusion located therein;

FIG. 5 is a side view of the distal end of the apparatus shown in FIG. 4, in which the distal end of the microcatheter is shown positioned within an artery of a patient's vasculature after being advanced into, or through, the occlusion;

FIG. 6 is a side view of the distal end of the apparatus shown in FIG. 5, in which the distal end of the microcatheter is shown positioned within an artery of a patient's vasculature with fragments of the occlusion held against an outer surface thereof under the influence of a source of a vacuum applied to the perforations, and in which the inflatable members are shown deflated prior to removal from the patient's vasculature;

FIG. 7 is a close-up side view of the distal end of the apparatus shown in FIG. 3 according to one variation thereof in which the perforations are moveable between an open configuration and a closed configuration, wherein the apparatus is shown in a relaxed state in which the perforations are in the closed configuration;

FIG. 8 is a close-up side view of the distal end of the apparatus shown in FIG. 7, wherein the apparatus is shown in an extended state in which the perforations are in the open configuration;

FIG. 9 is a close-up side view of the distal end of the apparatus shown in FIG. 7, wherein the apparatus is shown positioned within an artery of a patient's vasculature after being advanced into, or through, an occlusion such that the perforations are located radially adjacent alongside the occlusion, wherein the apparatus is shown in the relaxed state in which the perforations are in the closed configuration;

FIG. 10 is a close-up side view of the distal end of the apparatus shown in FIG. 9, wherein the apparatus is shown in the extended state positioned within an artery of a patient's vasculature with fragments of the occlusion held against an outer surface thereof under the influence of a source of a vacuum applied to the perforations;

FIG. 11 is a close-up side view of the distal end of the apparatus shown in FIG. 9, wherein the apparatus is shown within an artery of a patient's vasculature, wherein the apparatus has returned to a relaxed state in which the perforations have returned to the closed configuration while fragments of the occlusion are held against the outer surface of the apparatus, thereby grasping the fragments of the occlusion for removal from the patient's vasculature, and wherein the inflatable members are shown in a deflated state;

FIG. 12 is a perspective view of the distal end of the apparatus shown in FIG. 7 according to one variation thereof in which the microcatheter is provided with centrally-located aspiration lumen and an offset guidewire lumen;

FIG. 12A is a section view of the apparatus of FIG. 12 taken along section plane A-A of FIG. 12 in which section plane A-A intersects a longitudinal axis of the apparatus;

FIG. 13 is a perspective view of the distal end of the apparatus shown in FIG. 12 according to one variation thereof in which the guidewire lumen has a closed distal end;

FIG. 14 is a close-up cross-sectional side view of an apparatus according to an alternative embodiment of the present invention in which a microguidewire is provided within a pusher catheter that is positioned within an aspiration catheter;

FIG. 15 is a close-up cross-sectional side view of an apparatus according to an alternative embodiment of the present invention in which a microguidewire is provided with a stopper member positioned within a tubular member;

FIG. 15A is a close-up partial cross-sectional side view of an apparatus according to an alternative embodiment of the present invention in which a microcatheter is provided with a conical shaped distal end shoulder against which a mating tapered surface of a microguidewire abuts for providing distal extension of a portion of the apparatus;

FIG. 16 is a perspective view of an apparatus according to an alternative embodiment of the present invention in which the apparatus comprises an inner tubular member and an outer tubular member slidably connected to one another along a common longitudinal axis;

FIG. 17 is a side view of a distal end of the apparatus shown in FIG. 16 in which the apparatus is shown in a relaxed state;

FIG. 18 is a side view of a distal end of the apparatus shown in FIG. 16 in which the apparatus is shown in an extended state;

FIG. 19 is a side view of a distal end of the apparatus shown in FIG. 16 in which the apparatus is shown in a relaxed state and wherein the apparatus is shown with an optional extensible member;

FIG. 20 is a side view of a distal end of the apparatus shown in FIG. 16 in which the apparatus is shown in an extended state and wherein the apparatus is shown with an optional extensible member;

FIG. 21 is a perspective view of an apparatus according to an alternative embodiment of the present invention in which the apparatus comprises a tubular member comprising a flexible elongated proximal segment and a distal segment bonded to a distal end of the flexible elongated proximal segment;

FIG. 22 is a perspective view of an apparatus according to yet another embodiment of the present invention in which the apparatus comprises an outer tubular member and an inner tubular member moveable relative thereto, wherein the outer tubular member is provided with a perforated section located near a distal end thereof and is positioned between a distal inflatable member located on the inner tubular member near a distal end thereof and a proximal inflatable member located on the outer tubular member proximal to the perforated section;

FIG. 23A is a side view of the apparatus shown in FIG. 22 in which an extensible member is provided between the distal and proximal inflatable members and wherein the apparatus is shown in a relaxed state;

FIG. 23B is a side view of the apparatus shown in FIG. 23A wherein the apparatus is shown in an extended state;

FIG. 24A is a side view of the apparatus shown in FIG. 22 in which an extensible member is provided between the distal and proximal inflatable members and wherein the apparatus is shown in a relaxed state positioned within an artery of a patient's vasculature such that the perforated section is positioned radially adjacent alongside an occlusion;

FIG. 24B is a side view of the apparatus shown in FIG. 24A wherein the apparatus is shown in an extended stated and wherein fragments of the occlusion are shown held against an outer surface of the outer tubular member under the influence of a source of a vacuum applied to the perforated section;

FIG. 24C is a side view of the apparatus shown in FIG. 24B wherein the apparatus has returned to the relaxed state such that fragments of the occlusion held against the outer surface of the outer tubular member are grasped by the extensible member for removal from the patient's vasculature;

FIG. 25 is a section view of the apparatus of FIG. 22 taken along section plane B-B of FIG. 22 in which section plane B-B intersects a longitudinal axis of the apparatus;

FIG. 26 is a perspective view of an apparatus according to another embodiment of the present invention in which the apparatus comprises an outer tubular member and an inner tubular member moveable relative thereto, wherein the outer tubular member is provided with a perforated section located at a distal end thereof, wherein the inner tubular member comprises a distal inflatable member located near a distal end thereof and wherein an extensible member is shown in an extended state between the distal inflatable member and a proximal end of the perforated section;

FIG. 27A is a side view of the apparatus shown in FIG. 26 in which the apparatus is shown in a relaxed state positioned within an artery of a patient's vasculature such that the perforated section is positioned radially adjacent alongside an occlusion and wherein the extensible member is shown in a compressed state; and,

FIG. 27B is a side view of the apparatus shown in FIG. 27A wherein the apparatus is shown in an extended stated and wherein fragments of the occlusion are shown held against an outer surface of the outer tubular member under the influence of a source of a vacuum applied to the perforated section.

### DETAILED DESCRIPTION OF THE PREFERRED EMBODIMENTS

An extensible microcatheter 10 according to one embodiment of the present invention is shown in FIG. 1 and is sized and configured to be introduced into a patient's vasculature which includes the arteries of a patient's brain, the arteries surrounding a patient's heart, and any branch artery stemming therefrom. Conventional devices and methods for introducing the microcatheter 10 into the patient's vasculature are utilized, including using needles, introducer sheaths, etc. (not shown) to provide percutaneous access into, for instance, the patient's radial, ulnar or femoral artery, and from there, to the rest of the patient's vasculature. Furthermore, conventional devices, such as, for example, guidewires, steering catheters, etc. (not shown) are utilized to “steer” the microcatheter 10 through the patient's vasculature toward a preselected location therein, such as, for example, the site of a vascular occlusion, blockage, obstruction, or the like, OC (FIG. 4) present in, for example, a cerebral artery AT (FIG. 4) of the patients' brain.

Generally, the microcatheter 10 is flexible and may assume a straight, relaxed configuration, as shown in FIG. 1, in which microcatheter 10 extends along a longitudinal axis LA from a proximal end 10a to a distal end 10b, the proximal end 10a and the distal end 10b defining a “relaxed” length LR therebetween. Once the microcatheter 10 has been inserted into and advanced through the patient's vasculature, the proximal end 10a is positioned outside the patient's body, whereas the distal end 10b is positioned within the patient's vasculature such that the distal end 10b is located proximate the occlusion OC (FIG. 4) for treatment and/or removal. While distal end 10b is shown as having blunt tip, distal end 10b may be shaped in the form of a tapered cone, rounded front end or other, similar, non-blunt tip so as to facilitate better navigability through the patient's vasculature while minimizing trauma to tissue, etc.

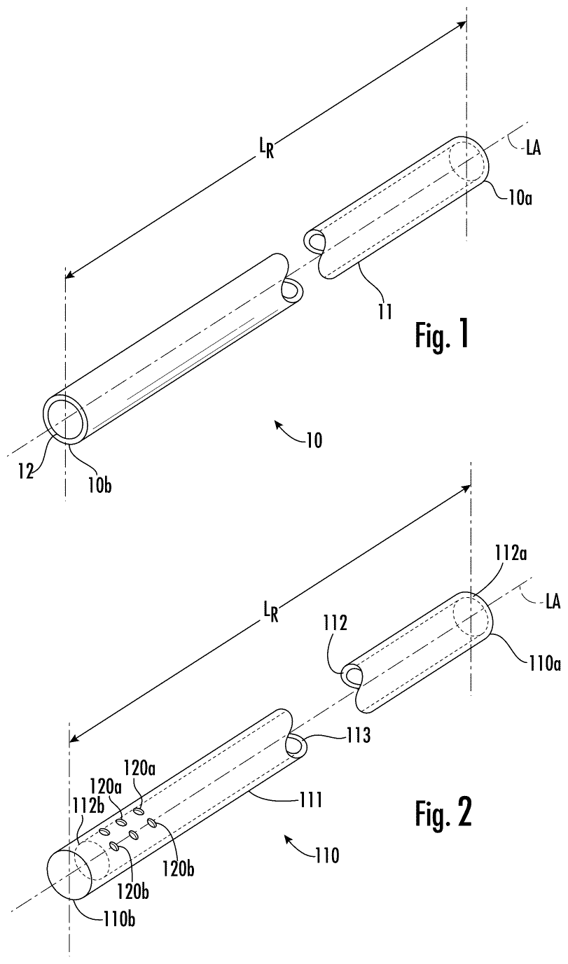

Microcatheter 10 includes a tubular member 11 having a lumen 12 which may be open at both the proximal end 10a and the distal end 10b of the microcatheter 10. Alternatively, lumen 12 may be closed at the distal end 10b, such as shown in FIG. 2. When, as shown in FIG. 1, lumen 12 is open at both the proximal end 10a and the distal end 10b, lumen 12 may provide a source of suction or aspiration, such as by vacuum or negative pressure system (not shown) connected to the proximal end 10a of the tubular member 11, such that the microcatheter 10 is adapted to suction the occlusion OC (FIG. 4) from the patient's vasculature through the open distal end 10b and into the lumen 12, thereby removing the occlusion from the patient's body. Alternatively, tubular member 11 may be connected at the proximal end 10a to a pressurized source of treatment solution, such as, for example, arterialized or oxygenated blood, thrombolytic agents, cold plasma, saline etc., to deliver the treatment solution via the lumen 12 through the open distal end 10b directly to the occlusion OC (FIG. 4). Even further, tubular member 11 may be connected at the proximal end 10a to an alternating, switchable source of suction and pressurized treatment solution such that lumen 12 is adapted to both deliver treatment solution to the occlusion OC (FIG. 4) and, when switched, aspirate the occlusion OC (FIG. 4), or fragments thereof, from the patient's body. When microcatheter 10 includes both open proximal and distal ends 10a, 10b, respectively, lumen 12 may provide a path through which a conventional microguidewire (not shown) may pass, for example, to assist in advancing the microcatheter 10 through the patient's vasculature and positioning the distal end 10b thereof proximate an occlusion OC (FIG. 4).

Referring now to FIG. 2, a microcatheter 110 according to one embodiment of the present invention is shown and is sized and configured to be introduced into the patient's vasculature utilizing conventional devices and methods, for example, needles, introducer sheaths, guidewires, steering catheters, etc. (not shown), configured to introduce the microcatheter 110 into and advance it through the patient's vasculature toward a preselected location therein, such as, for example, the site of a vascular occlusion OC (FIG. 4) present in, for example, a cerebral artery AT (FIG. 4) of a patients' brain.

Similar to the microcatheter 10 shown in FIG. 1, microcatheter 110 according to the embodiment shown in FIG. 2 is flexible and may assume a straight, relaxed configuration in which microcatheter 110 extends along a longitudinal axis LA from a proximal end 110a to a distal end 110b, the proximal end 110a and the distal end 110b defining a “relaxed” length L_{R} therebetween. Once the microcatheter 110 has been inserted into and advanced through the patient's vasculature, the proximal end 110a is positioned outside the patient's body, whereas the distal end 110b is positioned within the patient's vasculature such that the distal end 110b is located proximate the occlusion OC (FIG. 4) for treatment and/or removal.

Unlike the microcatheter 10 shown in FIG. 1, microcatheter 110 according to the embodiment shown in FIG. 2 includes a tubular member 111 with a lumen 112 having an open end 112a at the proximal end 110a of the microcatheter 110 but having a closed end 112b spaced proximally from the distal end 110b of the microcatheter 110. One of ordinary skill in the art will appreciate that the closed distal end 110b of the microcatheter 110 according to the present embodiment prevents the lumen 112 of the tubular member 111 to be utilized as a path through which a microguidewire (not shown) is used to assist in advancing the microcatheter 110 through the patient's vasculature and positioning the distal end 110b thereof proximate an occlusion OC (FIG. 4). Rather, one of ordinary skill in the art will appreciate that a microcatheter 110 according to the embodiment of the present invention shown in FIG. 2 must be advanced through the patient's vasculature utilizing known “blind” techniques, such as, for example, under X-ray (or similar) imaging in which the distal tip 110b of the microcatheter 110 is provided with a radiopaque coating or is constructed from a radiopaque material. Alternatively, a separate lumen (not shown) running alongside lumen 112 and formed in sidewall 113 may be open at both proximal and distal ends 110a, 110b, respectively, of the microcatheter 110 such that a microguidewire (not shown) can be advanced therethrough for the purpose of advancing, navigating and positioning the distal end 110b of the microcatheter 110 through a patient's vasculature. Alternatively still, a separate tubular member (not shown) with its own lumen (not shown) open at both ends may be positioned alongside and adhered to the tubular member 111, or integrally formed therealong such as, for example, by co-extrusion, such that a microguidewire (not shown) can be advanced therethrough for the purpose of advancing, navigating and positioning the distal end 110b of the microcatheter 110 through a patient's vasculature.

One or more perforations 120a, 120b are provided in a sidewall 113 of the tubular member 111 defined by the lumen 112, near the distal end 110b of the microcatheter 110, thereby allowing the lumen 112 to be in fluid communication with a region radially exterior to the sidewall 113 of the tubular member 111 near the distal end 110b of the microcatheter 110. Perforations 120a, 120b may include one or more perforations spaced both longitudinally (i.e., linearly along longitudinal axis LA) and annularly (i.e., angularly around longitudinal axis LA) from one another. The microcatheter 110 according to the present embodiment shows three sets of two perforations 120a, 120b spaced longitudinally along longitudinal axis LA in which each set of two perforations 120a, 120b includes a first perforation 120a located at the 12 o'clock angular position and a second perforation 120b located at the 3 o'clock angular position. Alternative arrangements include sets of perforations spaced radially equidistantly around longitudinal axis LA, such as, for example, sets of four perforations in which the perforations are positioned radially at the 12 o'clock, 3 o'clock, 6 o'clock, 9 o'clock angular positions, or any combinations thereof.

Lumen 112 may provide a source of suction or aspiration, such as by vacuum or negative pressure system (not shown) connected to the proximal end 110a of the tubular member 111 open to the open proximal end 112a of the lumen 112, such that the microcatheter 110 is adapted to provide a source of suction to the region radially exterior to the sidewall 113 of the tubular member 111. As such, if the distal end 110b of the microcatheter 110 is positioned such that the perforations 120a, 120b are proximate an occlusion OC (FIG. 4), suction may facilitate removal of the occlusion OC (FIG. 4), or fragments thereof, from the patient's vasculature.

Alternatively, tubular member 111 may be connected at the proximal end 110a to a pressurized source of treatment solution, such as, for example, arterialized or oxygenated blood, thrombolytic agents, cold plasma, saline etc., to deliver the treatment solution via the lumen 112 through the perforations 120a, 120b directly to the occlusion OC (FIG. 4). Even further, tubular member 111 may be connected at the proximal end 110a to an alternating, switchable source of suction and pressurized treatment solution such that lumen 112 is adapted to both deliver treatment solution to the occlusion OC (FIG. 4) and, when switched, aspirate the occlusion OC (FIG. 4), or fragments thereof, from the patient's body.

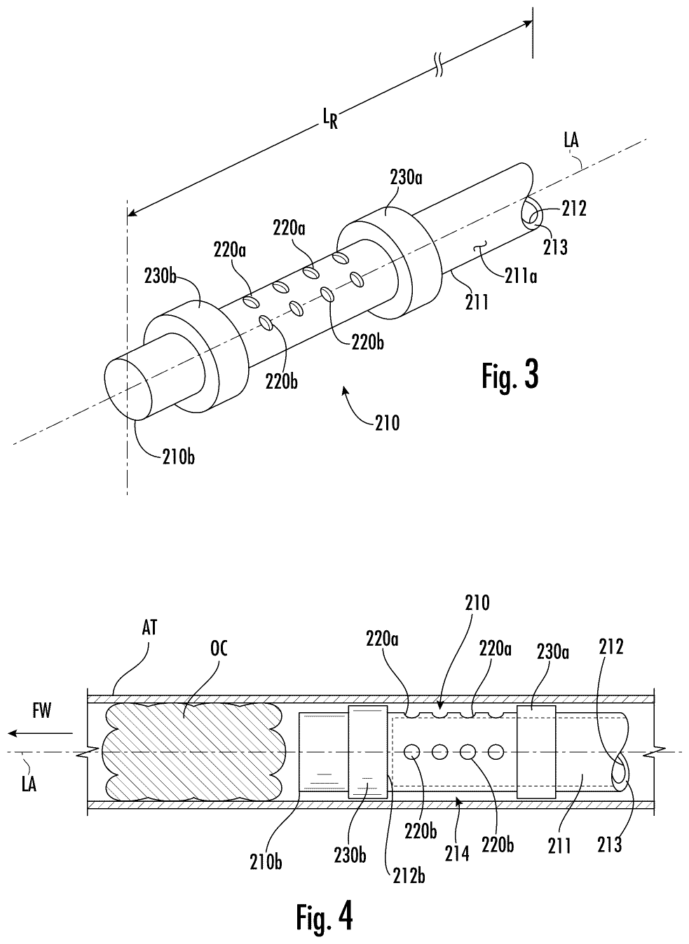

Referring to FIG. 3, a distal tip of a microcatheter 210 according to yet another embodiment of the present invention is shown and is sized and configured to be introduced into the patient's vasculature utilizing conventional devices and methods, for example, needles, introducer sheaths, guidewires, steering catheters, etc. (not shown), configured to introduce the microcatheter 210 into and advance it through the patient's vasculature toward a preselected location therein, such as, for example, the site of a vascular occlusion OC (FIG. 4) present in, for example, a cerebral artery AT (FIG. 4) of a patients' brain.

Similar to the microcatheter 110 shown in FIG. 2, microcatheter 210 according to the embodiment shown in FIG. 3 is flexible and may assume a straight, relaxed configuration in which microcatheter 210 extends along a longitudinal axis LA from a proximal end (not shown) to a distal end 210b, the proximal end (not shown) and the distal end 210b defining a “relaxed” length L_{R} therebetween. Once the microcatheter 210 has been inserted into and advanced through the patient's vasculature, the proximal end (not shown) is positioned outside the patient's body, whereas the distal end 210b is positioned within the patient's vasculature such that the distal end 210b is located proximate the occlusion OC (FIG. 4) for treatment and/or removal.

Similar to the microcatheter 110 shown in FIG. 2, microcatheter 210 according to the embodiment shown in FIG. 3 includes a tubular member 211 with a lumen 212 having an open end (not shown) at the proximal end (not shown) of the microcatheter 210 and having a closed end (not shown) spaced proximally from the distal end 210b of the microcatheter 210. One or more perforations 220a, 220b are provided in a sidewall 213 of the tubular member 211 defined by the lumen 212, near the distal end 210b of the microcatheter 210, thereby allowing the lumen 212 to be in fluid communication with a region 214 radially exterior to the sidewall 213 of the tubular member 211 near the distal end 210b of the microcatheter 210. Perforations 220a, 220b may include one or more perforations spaced both longitudinally (i.e., linearly along longitudinal axis LA) and annularly (i.e., angularly around longitudinal axis LA) from one another. The microcatheter 210 according to the present embodiment shows four sets of two perforations 220a, 220b spaced longitudinally along longitudinal axis LA in which each set of two perforations 220a, 220b includes a first perforation 220a located at the 12 o'clock angular position and a second perforation 220b located at the 3 o'clock angular position. Alternative arrangements include sets of perforations spaced radially equidistantly around longitudinal axis LA, such as, for example, sets of four perforations in which the perforations are positioned radially at the 12 o'clock, 3 o'clock, 6 o'clock, 9 o'clock angular positions, or any combinations thereof.

Lumen 212 may provide a source of suction or aspiration, such as by vacuum or negative pressure system (not shown) connected to the proximal end (not shown) of the tubular member 211 open to the open proximal end (not shown) of the lumen 212, such that the microcatheter 210 is adapted to provide a source of suction to the region radially exterior to the sidewall 213 of the tubular member 211. As such, if the distal end 210b of the microcatheter 210 is positioned such that the perforations 220a, 220b are proximate an occlusion OC (FIG. 4), suction may facilitate removal of the occlusion OC (FIG. 4), or fragments thereof, from the patient's vasculature.

Microcatheter 210 according to the embodiment shown in FIG. 3 further includes one or more inflatable members 230a, 230b, such as inflatable balloons, connected to a source of low pressure (not shown), such as, for example, a pre-filled syringe containing either air or saline, in fluid communication with inflatable members 230a, 230b by, for example, a lumen (not shown) provided in the sidewall 213 of the tubular member 211 extending longitudinally along, and spaced radially from, longitudinal axis LA. Alternatively, the source of low pressure (not shown) may be in fluid communication with inflatable members 230a, 230b by a separate tubular member (not shown) running longitudinally alongside an outer surface 211a of the tubular member 211, which such separate tubular member (not shown) may be affixed to the outer surface 211a of the tubular member 211, such as formed by a conventional co-extrusion manufacturing process.

According to a preferred embodiment, inflatable members 230a, 230b include a proximal inflatable member 230a expandable in a radial direction relative to the tubular member 211 and spaced longitudinally along longitudinal axis LA proximally of the one or more perforations 220a, 220b. Inflatable members 230a, 230b also include a distal inflatable member 230b expandable in a radial direction relative to the tubular member 211 and spaced longitudinally along longitudinal axis LA distally of the one or more perforations 220a, 220b toward the distal end 210b of the microcatheter 210. As such, perforations 220a, 220b are positioned longitudinally along longitudinal axis LA spaced between proximal inflatable member 230a and distal inflatable member 230b.

Referring now also to FIG. 4, the microcatheter 210 according to the embodiment of FIG. 3 is shown positioned within an artery AT of the patient's vasculature prior to passing through, or otherwise “crossing,” an occlusion OC present therein. As shown in FIG. 4, the occlusion OC is substantially blocking blood from flowing in direction FW. The distal end 210b of the microcatheter 210 is shaped to pass through, or otherwise cross, the occlusion, meaning that as the microcatheter 210 is advanced distally through the patient's vasculature, and more particularly, advanced in direction FW through the patient's vasculature along (and within) the patient's artery AT, the distal end 210b of the microcatheter 210 is shaped to penetrate the occlusion OC and to pass therethrough. Prior to passing through, or otherwise crossing, the occlusion OC, inflatable members 230a, 230b are in a deflated state, as shown in FIG. 4, and generally held against the outer surface 211a of the tubular member 211 so that the microcatheter 210 can pass through the occlusion with ease.

As described above, lumen 212 passes through tubular member 211 of microcatheter 210 and is open at a proximal end (not shown) thereof but closed at a distal end 212a thereof (i.e., lumen 212 does not extend all the way to distal end 210b of the microcatheter 210). A steerable guidewire (not shown) or similar device may be provided and positioned within lumen 212 to steer or otherwise direct the microcatheter 210 through the vasculature, and through the occlusion OC, using conventional devices and techniques. Lumen 212 extends distally along the longitudinal axis LA a sufficient distance so that it is open to the one or more perforations 220a, 220b provided in the sidewall 213 of the tubular member 210.

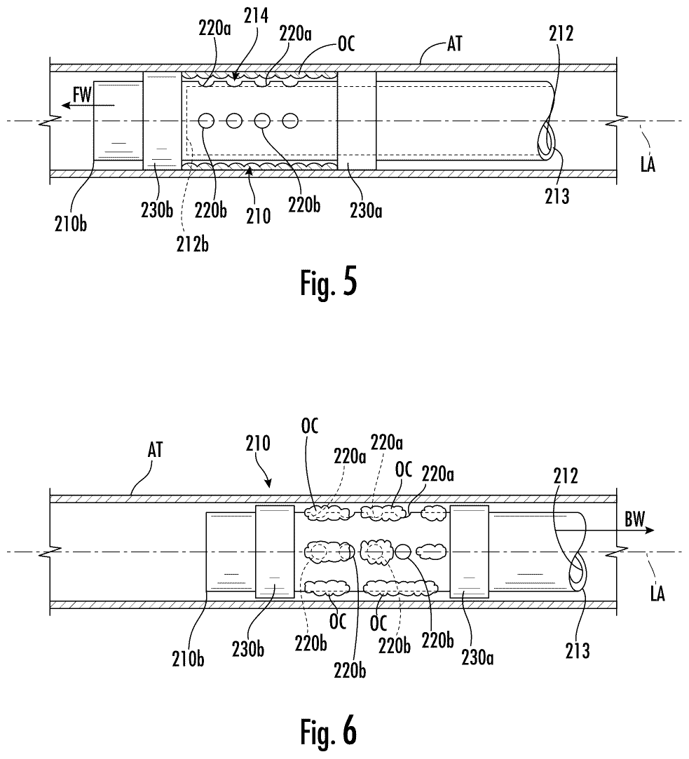

Referring now also to FIG. 5, the microcatheter 210 according to the embodiment of FIG. 3 and FIG. 4 is shown positioned within an artery AT of the patient's vasculature after the distal end 210b of the microcatheter 210 has been advanced in direction FW to pass through, or otherwise cross, occlusion OC. As shown in FIG. 5, the distal end 210b of the microcatheter 210 is advanced in direction FW until perforations 220a, 220b are positioned radially adjacent alongside the occlusion OC such that occlusion OC surrounds the outer surface 211 of the tubular member 211. Preferably, while in this position, occlusion OC is located fully between first and second inflatable members 230a, 230b, respectively, which are then inflated until they expand in a radial direction a sufficient distance to abut and rest against an inner surface of the artery AT. In this configuration, inflatable members 230a, 230b form a seal against the inner surface of the artery AT at both locations, thereby fully arresting blood flow in direction FW if the occlusion OC had not already previously arrested blood flow in this direction FW. More importantly, though, by forming a sealing fit against the inner surface of the artery AT, inflatable members 230a, 230b define an enclosed “chamber” 214 in a space at least partially occupied by the occlusion OC radially between the inner surface of the artery AT and the outer surface 211a of the tubular member 211, and longitudinally between first and second inflatable members 230a, 230b, respectively.

According to at least one embodiment of the present invention, lumen 212 and perforations 220a, 220b may be in fluid communication with a source (not shown) of treatment solution, such as, for example, arterialized or oxygenated blood, thrombolytic agents, cold plasma, saline etc., to deliver the treatment solution via the lumen 212 through the perforations 220a, 220b directly to the occlusion OC (FIG. 4) contained within the enclosed chamber 214. Treatment solution may be selected from among those known to one of ordinary skill in the art to be utilized to dissolve or otherwise disintegrate occlusions, obstructions or blockages such as occlusion OC.

With reference now also to FIG. 6, whether or not a treatment solution is used to partially or fully dissolve or disintegrate occlusion OC, lumen 212 may be connected at its proximal end (not shown) to the source of suction or aspiration, which is then activated to draw the occlusion OC, or fragments thereof, into the lumen 212 via the perforations 220a, 220b and out of the patient's body. Alternatively, once the source of suction has been activated, and after sufficient time has passed to allow the occlusion OC, or fragments thereof, to be drawn into the lumen 212 via perforations 220a, 220b and aspirated out of the patient's body, the inflatable members 230a, 230b can be deflated and the microcatheter 210 withdrawn from the patient's vasculature in a direction BW opposite direction FW. In an effort to remove as much of the occlusion OC as possible, source of suction may remain activated while the microcatheter 210 is being withdrawn from the patient's vasculature to thereby hold fragments of the occlusion OC against the outer surface 211a of the tubular member 211 as the microcatheter 210 is being withdrawn from the patient's vasculature, thereby enhancing the amount of the occlusion that is, in fact, removed from the patient's body.

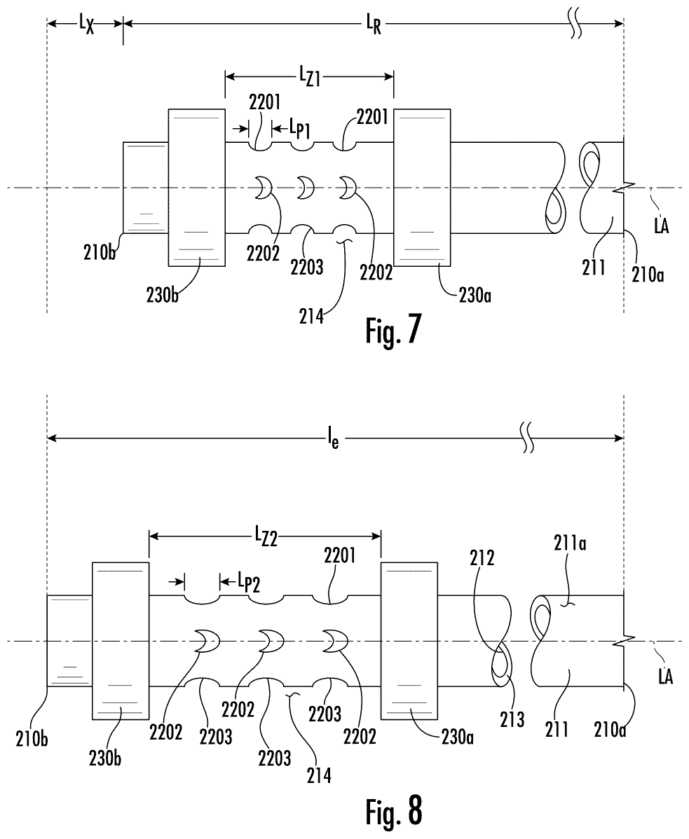

To further enhance the effectiveness of the microcatheter 210 to more fully and completely remove the occlusion OC from the patient's body, FIGS. 7 and 8 show an embodiment of the present invention in which tubular member 211 is formed from an extensible material, and preferably, a shape memory material, such as, for example, nitinol that is capable of elastically moving between a relaxed state (FIG. 7) and an extended state (FIG. 8). With specific reference to FIG. 7, tubular member 211 of microcatheter 210 is formed from a material that, when at rest, defines a first, relaxed length L_{R} extending between the proximal end 210a and the distal end 210b of the microcatheter 210. Referring now specifically to FIG. 8, tubular member 211 of microcatheter 210 is formed from a material that, when activated (as described in greater detail below), defines a second, extended length L_{E} that is greater than relaxed length L_{R} by an extension length L_{X}. Moreover, first and second inflatable members 230a, 230b, respectively, are spaced by a first distance L_{Z1} when the tubular member 211 is in the relaxed state (FIG. 7) and are spaced a second distance L_{Z2} greater than the first distance L_{Z1} when the tubular member 211 is in the extended state (FIG. 8).

If constructed from a shape memory material such as nitinol, and as will be described in even greater detail below, tubular member 211 may be manipulated to controllably move from the relaxed state (FIG. 7) to the extended state (FIG. 8), and then back again repeatedly, by applying (and removing) an external influence, such as, for example, heat, electricity, force, pressure, light, radio waves, etc., depending on the specific characteristics of the chosen material.

One or more perforations 2201, 2202, 2203 are provided in the sidewall 213 of the tubular member 211 defined by the lumen 212, near the distal end 210b of the microcatheter 210, thereby allowing the lumen 212 to be in fluid communication with a region 214 radially exterior to the sidewall 213 of the tubular member 211 near the distal end 210b of the microcatheter 210. Perforations 2201, 2202, 2203 may include one or more perforations spaced both longitudinally (i.e., linearly along longitudinal axis LA) and annularly (i.e., angularly around longitudinal axis LA) from one another. The microcatheter 210 according to the present embodiment shows three sets of three perforations 2201, 2202, 2203 spaced longitudinally along longitudinal axis LA in which each set of three perforations 2201, 2202, 2203 includes a first perforation 2201 located at the 12 o'clock angular position, a second perforation 2202 located at the 3 o'clock angular position and a third perforation 2203 located at the 6 o'clock angular position. Additional perforations (not shown) may be provided at longitudinal and angular positions between inflatable members 230a, 230b other than as shown in FIGS. 7 and 8.

As shown in FIG. 7, perforations 2201, 2203 may be configured such that when the tubular member 211 is in the relaxed state, the perforations 2201, 2203 have a first shape, such as a circle or oval with a relaxed midspan distance of L_{P1}. Additionally, perforations 2202 may be configured such that when the tubular member 211 is in the relaxed state, the perforations 2203 have a second shape, such as a crescent, arc or simple lateral slit. Any variety or arrangement of shapes may be used for perforations 2201, 2202, 2203 to achieve the purposes described in greater detail herein. As shown now in FIG. 8, perforations 2201, 2202, 2203 are configured such that when the tubular body 211 is manipulated to arrive to the extended state, perforations 2201, 2202, 2203 likewise achieve an “open” configuration such that, for example, they have an open midspan distance L_{P2} that is greater than relaxed midspan distance L_{P1} which, in effect, render perforations 2201, 2202, 2203 “larger” and more suitable to suction or aspirate greater volumes of occlusion OC (not shown) therethrough. It will be understood by one of ordinary skill in the art, upon reading the within disclosure, that perforations 2201, 2202, 2203, particularly perforations having a slit-like configuration, will enhance the physical extensibility of the microcatheter 210. That is, while the extensibility of microcatheter 210 according to certain embodiments hereof have been described with reference to the physical properties of the material used to construct the tubular member 211, one of ordinary skill in the art will appreciate that the size, shape, number, location and arrangement of the perforations 2201, 2202, 2203 will likely have an equal, if not greater, influence on the degree, direction and control of the extensibility thereof, as well as of the behavior of the microcatheter 210 to return to the relaxed state and to be repeatably, and reliably, extended and returned to its relaxed state.

With reference to FIGS. 9-11, certain advantages provided by the arrangement and configuration of perforations 2201, 2202, 2203 to perform a method according to an embodiment of the present invention will now be described. Referring specifically to FIG. 9, microcatheter 210 is advanced through the patient's vasculature and positioned proximate the occlusion OC, using the devices and techniques described herein, until the microcatheter 210 is positioned relative to the occlusion OC such that the occlusion OC is located between first and second inflatable members 230, 230b, respectively. Once in position, inflatable members 230a, 230b are then inflated to “trap” occlusion OC within the region 214 radially exterior to the sidewall 213 of the tubular member 211 near the distal end 210b of the microcatheter 210. While microcatheter 210 is being advanced and positioned within the patient's vasculature, tubular member 211 is in the relaxed state in which perforations 2201, 2202, 2203 are in the closed configuration.

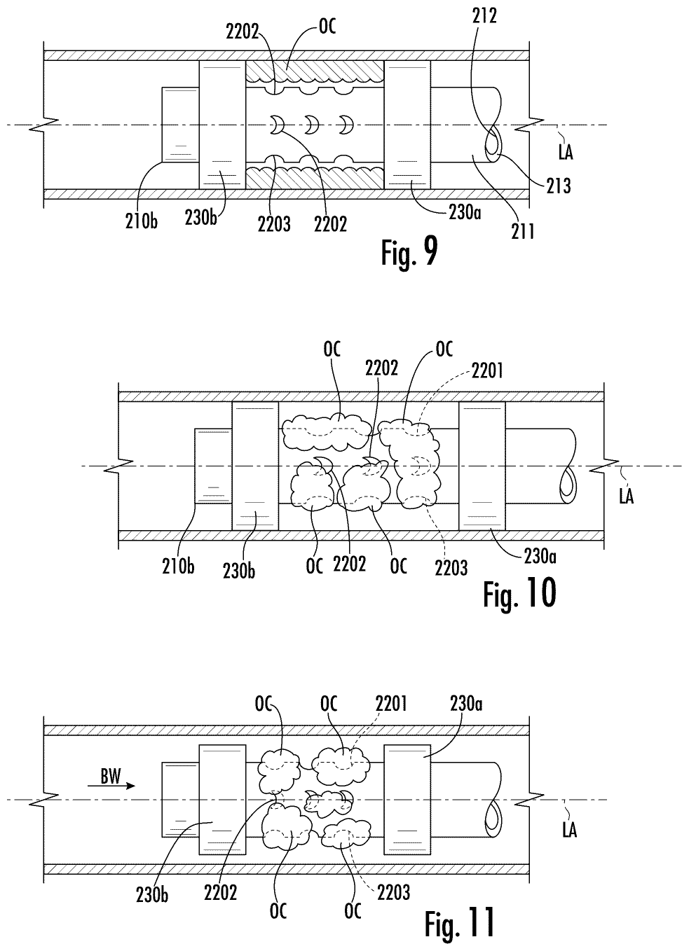

Referring now to FIG. 10, tubular member 211 is thereafter activated, causing it to achieve an extended state in which perforations 2201, 2202, 2203 are in the open configuration. In such an open configuration, for example, perforations 2201, 2203 assume an oval shape (rather than a circular shape, such as when they are in the relaxed configuration) elongated along the longitudinal axis LA. In addition, perforations 2202, assume an open-mouth configuration when the tubular member 211 is in the extended state, rather than a relaxed-mouth configuration such as when the tubular member 211 is in the relaxed state. Source of suction (not shown) is thereafter activated, creating a negative pressure within lumen 212, thereby drawing occlusion OC, or fragments thereof, toward the outer surface 211a of the tubular member 211 and trapped deep within the open perforations 2201, 2202, 2203. Source of suction remains activated for a sufficient period of time to permit some, if not all, fragments of the occlusion OC to be aspirated and withdrawn entirely from the patient's body via lumen 212.

Referring now to FIG. 11, while source of suction remains activated, the extensible tubular member 211 (which is in the extended state) is deactivated causing it to, under its own elastic material properties, return to the relaxed state, thereby causing perforations 2201, 2202, 2203 to return to their closed configurations, however, now while portions or fragments of occlusion OC are held therein. Perforations 2201, 2202, 2203, then, physically capture, grasp, grip, hold or retain the occlusion, or fragments thereof, against the outer surface 211a of the tubular member 211 of the microcatheter 210, thereby enhancing removal of the occlusion OC from within the patient's vasculature while the microcatheter 210 is removed therefrom (taking the occlusion OC with it). Again, any size, shape or arrangement of perforations 2201, 2202, 2203 can be selected to optimize both their ability to open as the tubular member 211 moves from a relaxed state to an extended state and their ability to physically capture the occlusion OC, or fragments thereof to facilitate a more full and complete removal of the occlusion OC from the patient's body once the tubular member 211 is returned to the relaxed state. In addition, features (not shown), such as teeth, spikes, grooves, or the like, may be provided extending at least partially into any one of the openings of any one of the perforations 2201, 2202, 2203 to even further enhance their ability to physically grasp the occlusion OC, or fragments thereof once the occlusion OC, or fragments thereof, have been withdrawn into the perforations 2201, 2202, 2203 as described herein.

Moreover, while perforations 2201, 2202, 2203 have heretofore been described as extending completely through the sidewall 213 of the tubular member 211 (thereby defining perforations 2201, 2202, 2203 as a source of aspiration of the occlusion OC out of the patient's body via the lumen 212), some or all of the perforations 2201, 2202, 2203 may be formed as only surface features, i.e., they do not extend completely through the sidewall 213 of the tubular member 211, but instead form physical grooves, dimples or pockets in the outer surface 211a of the tubular member 211. Such perforations 2201, 2202, 2203 formed as mere surface features would be function as a source of aspiration of the occlusion OC out of the patient's body via the lumen 212, however, they may be sufficient to physically capture, grasp, grip, hold or retain the occlusion, or fragments thereof, against the outer surface 211a of the tubular member 211 of the microcatheter 210. Perforations 2201, 2202, 2203, and any features thereof, may be formed in the tubular member 211 by any conventional technique, including machining, laser cutting, etc.

Material for tubular member 211 is selected to provide inherent sufficient elasticity and stiffness to enhance the ability of the perforations 2201, 2202, 2203 to open and close as described herein for the purpose of physically grasping the occlusion OC, or fragments thereof, when the tubular member 211 is returned to its relaxed state after engaging the occlusion. And while the inherent elastic properties of the material used to form tubular member 211 is important, the presence, size, shape and arrangement of the perforations 2201, 2202, 2203, themselves, may provide, or add to, the compounded elastic properties of the tubular member 211. In other words, elasticity of the tubular member 211 may be provided either by the inherent elastic properties of the material selected, may be provided solely by the present, size, shape and arrangement of the perforations 2201, 2202, 2203 formed therein, or may be provided by any combination of the foregoing. Indeed, a tubular member 211 constructed from a substantially inelastic material could be rendered highly elastic solely by one or more specific combinations of size, shape and arrangement of the perforations 2201, 2202, 2203.

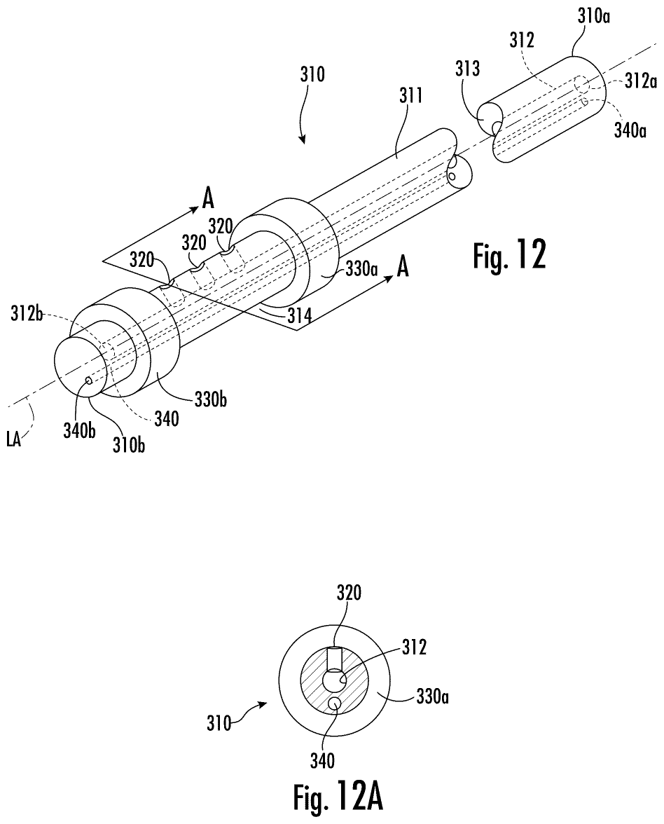

Various other embodiments of the present invention will now be described with reference to the embodiments described previously in which like reference numerals are intended to represent like features, components or aspects of the present invention. For example, referring now to FIGS. 12 and 12A, a microcatheter 310 according to an embodiment of the present invention is shown in a simplified configuration for illustration purposes and includes a proximal end 310a and a distal end 310b. In this embodiment, microcatheter 310 includes a tubular member 311 having a central lumen 312 extending along a longitudinal axis LA of the microcatheter 310 and defined by an open proximal end 312a and a closed distal end 312b. A sidewall 313 of the tubular member 311 is defined by the central lumen 312. A guidewire lumen 340 extends along the longitudinal axis LA radially spaced from and parallel to central lumen 312 and is defined by an open proximal end 340a and an open distal end 340b. Guidewire lumen 340 may be used with a conventional guidewire (not shown) to assist in advancing the microcatheter 310 through a patient's vasculature to pass through, or otherwise cross, an occlusion (not shown) present in a patient's vasculature and to position the distal end 310b of the microcatheter 310 proximate the occlusion (not shown) for treatment and/or removal from the patient's body as described herein with reference to the various embodiments of the present invention.

One or more inflatable members 330a, 330b are provided near the distal end 310b of the microcatheter 310 and are connected to a source (not shown) of pressurized fluid, such as a syringe filled with air or saline, by a separate lumen (not shown) to controllably inflate and deflate the inflatable members 330a, 330b as described above with respect to various other embodiments of the present invention. One or more perforations 320 are provided in the sidewall 313 of the tubular member 313 between inflatable members 330a, 330b and extend radially therethrough such that they are in fluid communication with central lumen 312. A source of suction (not shown), such as by vacuum or negative pressure system, may be connected to the proximal end 312a of central lumen 312, thereby providing a source of suction or aspiration to a region 314 radially exterior to the sidewall 313 of the tubular member 311 near the distal end 310b of the microcatheter 310. The number, size, shape and arrangement of perforations 320 may by adjusted to optimize performance of the microcatheter 310 for the purposes, and to achieve the objectives, described herein. With reference now also to FIG. 12A, microcatheter 310 according to an embodiment of the present invention is depicted in which perforations 320 are shown in the 12 o'clock angular position and guidewire lumen 340 is shown in the 6 o'clock angular position. Guidewire lumen 340 may be located at any angular position around longitudinal axis selected to avoid perforations 320, etc., as the guidewire lumen 340 extends longitudinally alongside the longitudinal axis LA, thereby preventing the open distal end 340b of the guidewire lumen 340 from decreasing the amount of vacuum supplied to the region 314.

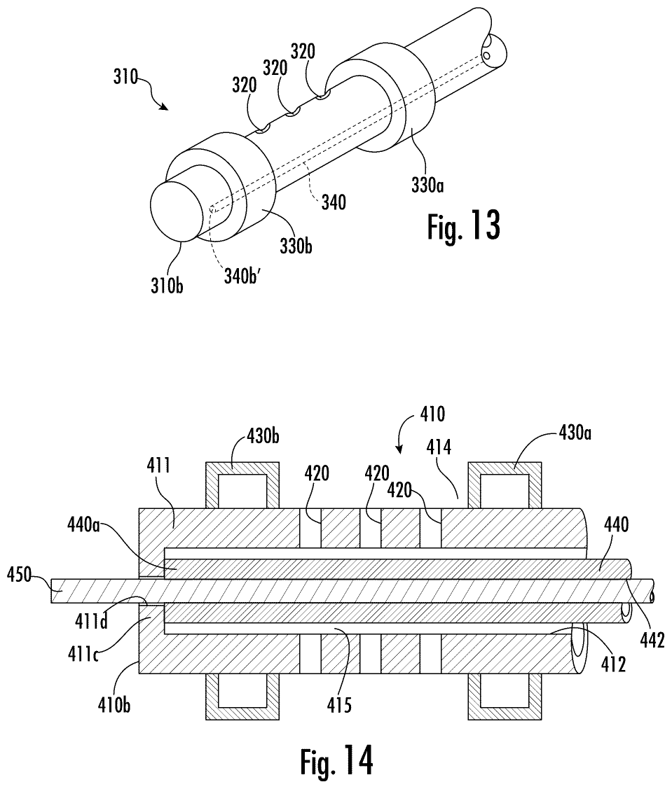

FIG. 13 depicts a microcatheter 310 according to the embodiment of the present invention shown in FIGS. 12 and 12A, modified slightly such that guidewire lumen 340 does not extend all the way to the distal end 310b of the microcatheter 310, i.e., guidewire lumen 340 has a closed distal end 340b′ spaced proximally from the distal end 310b of the microcatheter 310. As modified, guidewire lumen 340 is not used, as described above with reference to FIGS. 12 and 12A, but rather is used to extend microcatheter 310 from a related state to an extended state, as described in more detail above. Specifically, a microguidewire (not shown) is advanced through the guidewire lumen 340, from the open proximal end 340a (FIG. 12) thereof until a distal tip (not shown) of the microguidewire abuts the closed distal end 340b′. Applying a distal force to the microguidewire while holding the proximal end 310a (FIG. 12) of the microcatheter 310 will effectively extend the tubular member 311 axially from the relaxed length LR to the extended length LE as described herein more specifically with reference to FIGS. 7 and 8, a sufficient distance to open perforations as described above. Tubular member 311 is constructed from a sufficiently elastic material to permit its extension under such force. Further, microguidewire is constructed from a sufficiently stiff material to permit its applying an axial force against the closed distal end 340b to extend the tubular member 311 and open the perforations 320 thereby.

Providing a side-loaded guidewire lumen 340 (i.e., offset radially from the central lumen 312) with a closed distal end 340b also provides distinct advantages regarding the ability of the microcatheter 310 to navigate the tortuous pathway of a patient's vasculature, especially the narrow arteries of the brain. For example, pre-loading a steerable microguidewire in the guidewire lumen 340 prior to inserting the microcatheter 310 in the patient's vasculature provides enhanced axial strength/stiffness which aids in navigability, especially in tortuous or diseased vasculature. Alternatively, the microguidewire might be inserted into the guidewire lumen 340 only after the microcatheter 310 has been advanced within the patient's vasculature far enough to encounter tortuous or diseased portions thereof.

With reference to FIG. 14, a microcatheter 410 is shown according to one embodiment of the present invention in which microcatheter 410 is formed from a tubular member 411 that is configured similarly to tubular members 11, 111, 211 and 311 according to various embodiments hereof. Tubular member 411 according to the present embodiment defines a lumen 412 extending between an open proximal end (not shown) and an open distal end 410b of the microcatheter 410. Proximal and distal inflatable members 430a, 430b, respectively, are provided near the distal end 410b of the microcatheter 410 and are configured to be controllably inflated and deflated similar to embodiments of the present invention described elsewhere herein. Perforations 420 are provided in the tubular member 411 such that a source of vacuum or aspiration connected to the proximal end (not shown) of the microcatheter 410 provides aspiration to a region 414 radially outwardly from an exterior surface of the tubular member 411 through the lumen 412 via the perforations 420.

A pusher catheter 440 is positionable within the lumen 412 and is slidable relative thereto. Pusher catheter 440 has an outer diameter that is smaller than an inner diameter of lumen 412 of the tubular member 411 such that an annular spaced chamber 415 is defined therebetween through which source of vacuum (not shown) communicates with the exterior region 414 via perforations 420 when pusher catheter 440 is positioned within the lumen 412 of the tubular member 411.

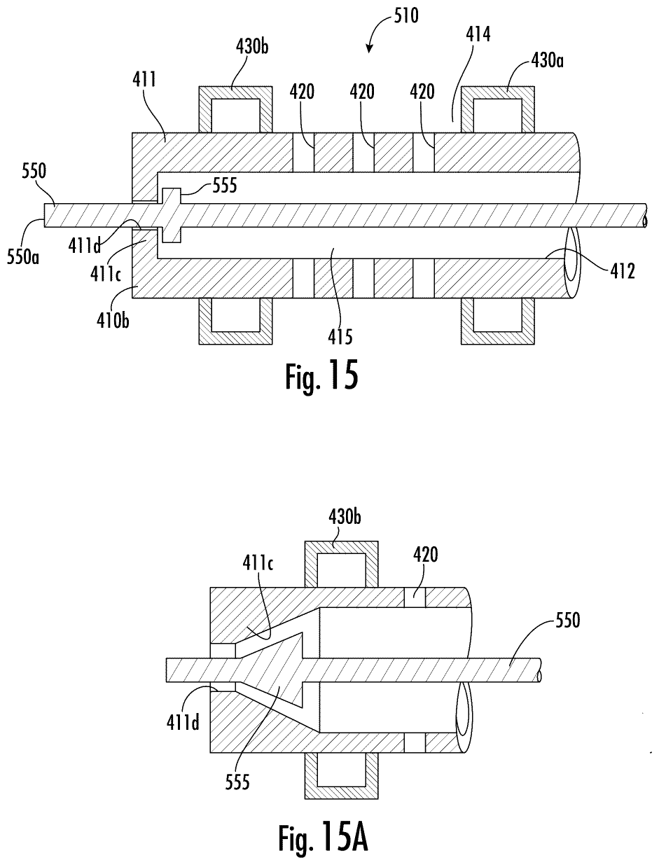

Distal end 410b of the microcatheter 410 defines a radially inwardly-directed shoulder 411c further defining an opening 411d through the distal end 410b communicating with lumen 412. In FIG. 15, shoulder 411c is shown to have a flat radial blunt face the surface of which forms a right angle with the inner surface of the lumen 412, but as shown in FIG. 15A, shoulder 411c may form an angle with the inner surface of the lumen 412, in which case, shoulder 411c provides an internal conical surface area.

Referring back to FIG. 15, pusher catheter 440 has a distal end 440a with an outer diameter that is larger than an inner diameter of opening 411d such that proximal advancement of the pusher catheter 440 within the tubular member 411 abuts the distal end 440a of the pusher catheter 440 against the shoulder 411c. Pusher member 440 is constructed from a sufficiently stiff material such that proximal force applied to the pusher member 440 when the distal end 440a thereof abuts the shoulder 411c, while holding the proximal end (not shown) of the microcatheter 410, results in extending the microcatheter 410 from a relaxed state to an extended state as described in greater detail herein. Perforations 420 are sized, shaped and configured as described elsewhere herein such that they are adaptable to enhance the microcatheter's 410 ability to remove an occlusion OC from within a patient's vasculature as described herein and shown particularly with reference to the embodiment described and shown in FIGS. 9-11.

Pusher catheter 440 includes an inner lumen 442 through which a microguidewire 450 is positionable and movable therein. Opening 411d in tubular member 411 of the distal end 410b of the microcatheter 410 is sized such that the microguidewire 450 is permitted to pass therethrough. The microcatheter 410 according to the present embodiment provides an advantage of allowing for microguidewire-assisted navigability through the patient's vasculature while also providing for extensibility thereof, such as, for example, to move the perforations 420 from a relaxed configuration, such as shown in FIG. 14, to an open configuration (not shown) for the purposes described elsewhere herein. Moreover, the microcatheter 410 according to the present embodiment also provides an advantage of providing a sealed inner chamber 415 to enhance the use of vacuum force applied thereto.

With reference now to FIG. 15, a microcatheter 510 according to another embodiment of the present invention is shown having many components in common with the embodiment hereof shown in FIG. 14. Unlike the embodiment shown in FIG. 14, the microcatheter 510 of the present embodiment does not include a pusher catheter 440 (FIG. 14). Rather, microcatheter 510 includes a microguidewire 550 with a stopper member 555 formed near the distal end 550a thereof and positionable within the lumen 412 of the tubular member 411. Stopper member 555 may be integrally-formed with the microguidewire 550 or may be a separately inflatable member, such as a balloon (in which case, microguidewire 550 would further include an internal lumen (not shown) to provide a means by which stopper member 555 is inflated. The stopper member 555 is positioned axially proximally of the distal end 550a of the microguidewire 550 such that distal advancement thereof against the shoulder 411c, while at the same time holding the proximal end (not shown) of the microcatheter 510, effects extension of microcatheter 510 as described in greater detail elsewhere herein. Referring for a moment back to FIG. 15A, in the event shoulder 411c forms a tapered, conical inner surface area, stopper member 555 may have a similar mating conical outer surface configured to more efficiently engage the abutting tapered surface of shoulder 411c.

Referring now also to FIG. 15, withdrawing microguidewire 550 proximally such that stopper member 555 is spaced proximally from shoulder 411c by a small distance allows for vacuum to be delivered via lumen 412 through the opening 411d in shoulder 411c of the tubular member 411. Alternatively, if lumen 412 is connected to a pressurized source of treatment solution used, for example, to dissolve or partially dissolve an occlusion, such treatment solution can be supplied both to the region 414 immediately surrounding the perforations 420, but also through the distal end 410b of the microcatheter 510 through opening 411d. Microguidewire 550 may include a lumen (not shown) through which bypass blood or a treatment solution might be provided distally of the microcatheter as described elsewhere herein.

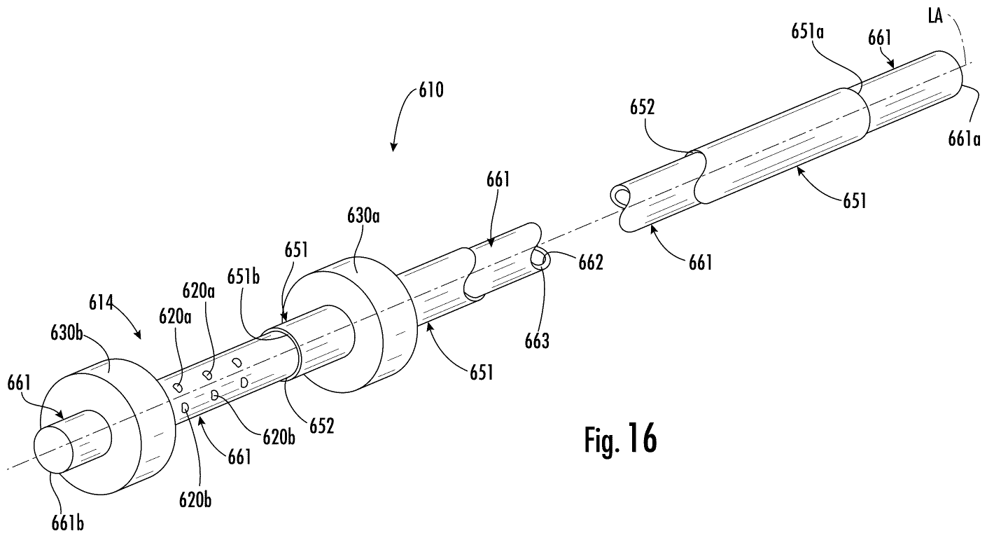

Referring to FIG. 16, a microcatheter 610 according to yet another embodiment of the present invention is shown having several components in common with the embodiments hereof previously described. Unlike the previous embodiments, though, the microcatheter 610 of the present embodiment comprises an outer tubular member 651 and an inner tubular member 661 slidably and rotatably positionable within a lumen 652 of the outer tubular member 651.

Inner tubular member 661 includes a lumen 662 therein extending along the longitudinal axis from a proximal end 661a to a closed distal end 661b. One or more perforations 620a, 620b are provided in a sidewall 663 of the inner tubular member 661 defined by the lumen 662 near the distal end 661b thereof. Perforations 620a, 620b may include one or more openings in the sidewall 663 spaced both longitudinally (i.e., linearly along longitudinal axis LA) and annularly (i.e., angularly around longitudinal axis LA) from one another. The microcatheter 610 according to the present embodiment shows three sets of two perforations 620a, 620b spaced longitudinally along longitudinal axis LA in which each set of two perforations 620a, 620b includes a first perforation 620a located at the 12 o'clock angular position and a second perforation 620b located at the 3 o'clock angular position. Alternative arrangements include sets of perforations spaced radially equidistantly around longitudinal axis LA, such as, for example, sets of four perforations in which the perforations are positioned radially at the 12 o'clock, 3 o'clock, 6 o'clock, 9 o'clock angular positions, or any combinations thereof.

Outer tubular member 651 includes an open proximal 651a and an open distal end 651b defining an opening through which distal end 661b of the inner tubular member 661 may pass from within the lumen 652. Advancing the inner tubular member 661 distally such that the distal end 661b thereof extends beyond the distal end 651b of the outer tubular member 651 exposes the perforations 620a, 620b in the inner tubular member 661 to a region 614 radially exterior to the sidewall 663 of the inner tubular member 661 distally of the distal end 651b of the outer tubular member 651. A source of suction (not shown) may be provided, such as by vacuum or negative pressure system, in communication with the perforations 620a, 620b via the lumen 662 to capture grasp, grip, hold or retain an occlusion OC, or the fragments thereof, against the sidewall 663 of the inner tubular member 661 of the microcatheter 610 as the microcatheter 610 is removed from the patient's vasculature, thereby more fully and completely removing the occlusion OC therewith. Alternatively, a source of treatment solution (not shown) may be provided under nominal pressure in communication with the perforations 620a, 620b via the lumen 662 to deliver treatment solution to the region 614 for the purpose of, for example, dissolving or partially dissolving an occlusion OC.

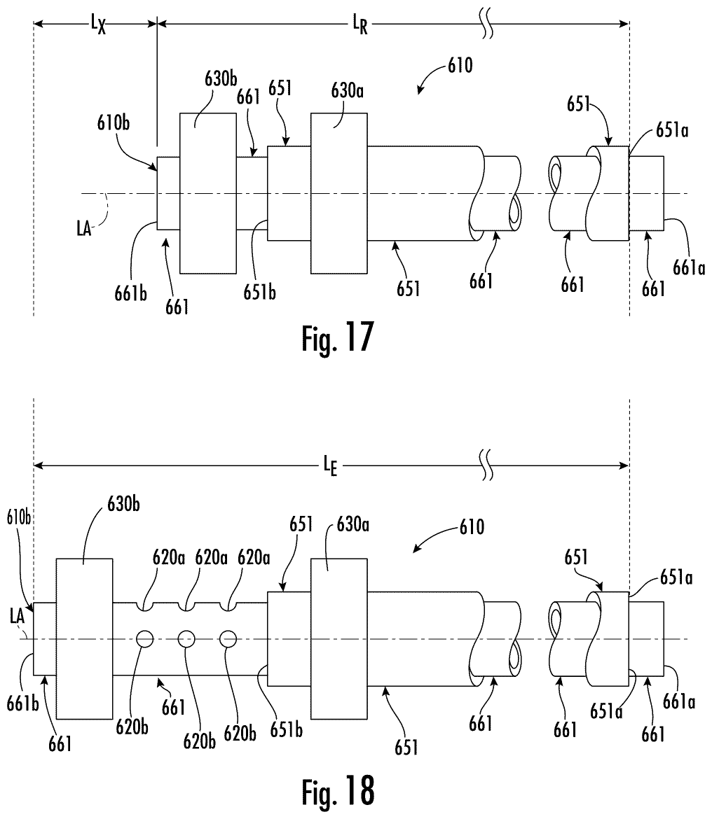

With additional reference now to FIGS. 17 and 18, microcatheter 610 is flexible and may assume a straight, relaxed configuration in which inner and outer tubular members 661, 651, respectively, of microcatheter 610 extend along the longitudinal axis LA such that a length between the proximal end 651a of the outer tubular member 651 and the distal end 661b of the inner tubular member defines a relaxed length L_{R} of the microcatheter 610 therebetween when inner tubular member 661 is at least partially withdrawn into the outer tubular member 651. Advancing the inner tubular member 661 distally relative to the outer tubular member 651 along the longitudinal axis LA increases the length between the proximal end 651a of the outer tubular member 651 and the distal end 661b of the inner tubular member, thereby defining an extended length L_{E} of the microcatheter 610 that is greater than the relaxed length L_{R} by an extension length L_{X}.

A method whereby the microcatheter 610 according to the present embodiment is used includes a step of introducing the microcatheter 610 into the patient's vasculature utilizing conventional devices and methods, for example, needles, introducer sheaths, guidewires, steering catheters, etc. (not shown), and advancing it through the patient's vasculature toward a preselected location therein, such as, for example, the site of a vascular occlusion OC present in, for example, a cerebral artery AT of a patients' brain. The method further includes advancing the microcatheter through the patient's vasculature while the microcatheter is in the “relaxed” state such that the inner tubular member 661 is withdrawn at least partially into the outer tubular member 651 and the proximal end 651a of the outer tubular member 651 is spaced from the distal end 661b of the inner tubular member along the longitudinal axis by the relaxed length L_{R}. The method even further includes advancing the microcatheter 610 through the patient's vasculature until the distal end 610b thereof is positioned within the patient's vasculature such that the distal end 610b is located proximate the occlusion OC for treatment and/or removal. Once the distal end 610b of the microcatheter 610 is located proximate the occlusion OC, the inner tubular member 661 is advanced distally relative to the outer tubular member 651, thereby exposing the perforations 620a, 620b to the occlusion OC. Once exposed, treatment solution may be delivered to the occlusion OC via perforations 620a, 620b under low pressure and/or vacuum or negative pressure supplied through perforations 620a, 620b for the purpose of removing the occlusion OC, or fragments thereof, from within the patient's vasculature.

Similar to certain other embodiments described herein, for example, the microcatheter 210 shown in FIGS. 3-11, microcatheter 610 according to the present embodiment may include one or more inflatable members 630a, 630b, such as inflatable balloons, connected, either individually or together, to a source of low pressure (not shown), such as, for example, a pre-filled syringe containing either air or saline, in fluid communication with inflatable members 630a, 630b by, for example, a lumen (not shown) provided in the sidewalls of the outer and inner tubular members 651, 661, respectively, extending longitudinally along, and spaced radially from, longitudinal axis LA. Alternatively, the source of low pressure (not shown) may be in fluid communication with inflatable members 630a, 630b by one or more separate tubular members (not shown) running longitudinally alongside an outer surface of either the outer or inner tubular members 651, 661, respectively.

According to the present embodiment, inflatable members 630a, 630b include a proximal inflatable member 630a affixed to the outer tubular member 651 near the distal end 651b thereof and expandable in a radial direction relative thereto. Inflatable members 630a, 630b also include a distal inflatable member 630b affixed to the inner tubular member 661 near the distal end 661b thereof and expandable in a radial direction relative thereto and spaced longitudinally along longitudinal axis LA distally of the one or more perforations 620a, 620b toward the distal end 610b of the inner tubular member 661. As such, when the inner tubular member 661 is extended distally relative to the outer tubular member 651 such that perforations 620a, 620b are exposed, perforations 620a, 620b are positioned longitudinally along longitudinal axis LA spaced between proximal inflatable member 630a and distal inflatable member 630b.

The microcatheter 610 of the present embodiment shows the inflatable members 630a, 630b in an expanded, inflated state. One of ordinary skill in the art will understand, upon reading the present disclosure, and in particular, as the present disclosure relates to the various other embodiments hereof, that inflatable members 630a, 630b may be deflated, individually or simultaneously, to reach a deflated state (not shown). Moreover, distal inflatable member 630b may include a profile that permits it to be withdrawn into the open distal end 651b of the outer tubular member 651, and more particularly, to be withdrawn into the lumen 652 that defines the open distal end 651b of the outer tubular member 651 when the distal inflatable member 630b is in the deflated state.

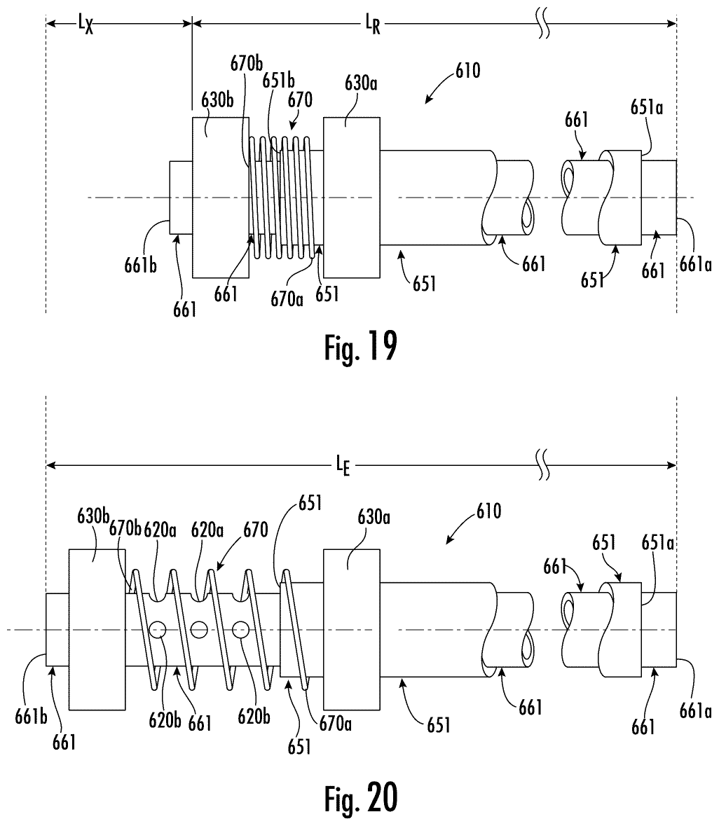

With reference now also to FIGS. 19 and 20, the microcatheter 610 according to the present invention is shown with an optional extensible member 670 affixed at a proximal end 670a thereof to the outer tubular member 651 near the distal end 651b thereof, and affixed at a distal end 670b thereof to the inner tubular member 661 near the distal end 661b thereof. According to one alternative of the present embodiment, extensible member 670 is shown as a compression spring-like coil structure with multiple loops, winds or coils wrapped around tubular members 651, 661 along the longitudinal axis LA of the microcatheter 610 between the proximal and distal ends 670a, 670b, respectively, of the extensible member 670. Extensible member 670 is configured to have a compressed state, as shown in FIG. 19, when the inner tubular member 661 is withdrawn into the outer tubular member 651, and configured to have an extended state, as shown in FIG. 20, when the inner tubular member 661 is extended distally relative to the outer tubular member 651 such that perforations 620a, 620b are exposed.

As shown in FIG. 19, when the extensible member 670 is in the compressed state, individual coils thereof are spaced such that they are separated from one another by only a small distance, or alternatively, they may be touching one another. And as shown in FIG. 20, extensible member 670 is configured such that when it is in the extended state, individual coils thereof are separated from one another by a distance suitable to enmesh an occlusion OC, or portions thereof, when the microcatheter 610 is advanced through a patient's vasculature such that, when the inner tubular member 661 is advanced distally relative to the outer tubular member 651 to expose the perforations 620a, 620b, the perforations 620a, 620b are positioned radially adjacent alongside the occlusion OC. Engagement of the individual coils of the extensible member 670 into the occlusion OC, or portions thereof, can be enhanced by applying a negative pressure to the perforations 620a, 620b via lumen 662 such that the occlusion OC is drawn toward the surface of the inner tubular member 661 by the negative pressure, thereby digging the individual coils of the extensible member 670 deep into the occlusion OC. At this point, the inner tubular member 661 can be manually advanced and retracted small distances repeatedly relative to the outer tubular member 651 so as to manipulate the individual coils of the extensible member 670 and dig them even deeper into the occlusion OC, thereby capturing, grasping, gripping, holding, retaining, entrapping and enmeshing the coils firmly into the occlusion OC.

Alternatively, the ability of the individual coils of the extensible member 670 to grasp the occlusion OC can be achieved according to the following, alternative, method of use in which the inner tubular member 661 is held in a fixed position, longitudinally relative to the patient's artery (and the occlusion OC located therein), and the outer tubular member 651 is manually advanced and retracted small distances repeatedly relative to the inner tubular member 661. Once positioned near the occlusion, the inner tubular member 661 is extended as described above to extend and expose the individual coils of the extensible member 670 to the occlusion OC). Distal inflatable member 630b may then be inflated, thereby softly anchoring the inner tubular member 661 (to which the distal inflatable member 630b is affixed) to the inner surface of the patient's artery proximate the distal-most portion of occlusion OC. Without inflating the proximal inflatable member 630a, outer tubular member 651 may be manually advanced and retracted small distances repeatedly relative to the inner tubular member 661 in much the same manner, and for the same purpose, as described above.

To be clear, by referring to the ability of the extensible member 670 to “dig into” the occlusion OC, what is meant is that, according to one embodiment of the present invention, the occlusion OC, or portions thereof, may be drawn into the space between successive coils of the extensible member 670, either under the influence of a negative pressure applied to the perforations 620a, 620b or naturally as a consequence of the coils of the extensible member 670 simply being physically close to, and embedded within, the occlusion OC, such that one or more of the individual coils of the extensible member 670 is embedded within the occlusion OC. Alternatively, expanding the extensible member 670 might result in the individual coils thereof being advanced into the tissue of the occlusion OC, or portions thereof.

Prior to withdrawing the microcatheter 610, along with the occlusion, or portions thereof, the inner tubular member 661 may be forcibly withdrawn proximally relative to the outer tubular member 651, either while a negative pressure remains applied to the perforations 620a, 620b or not, thereby causing the individual coils of the extensible member 670 to collapse on themselves, thereby physically grasping the occlusion OC even more firmly, enhancing the ability of the microcatheter 610 to remove the occlusion OC.

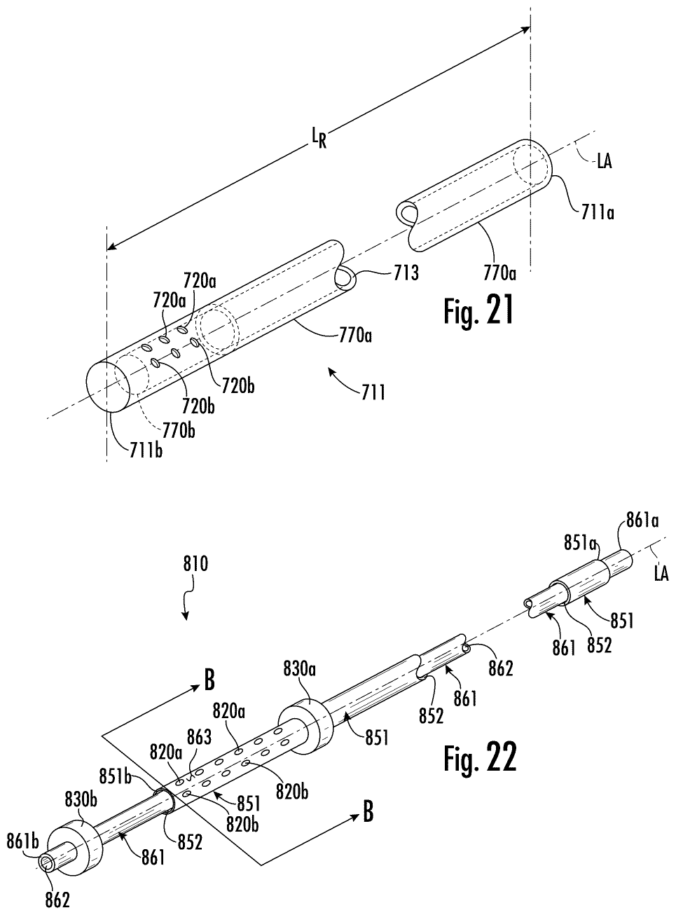

With reference to FIG. 21, a tubular member 711 according to one variation thereof that is compatible with any one the microcatheters 10, 110, 210, 310, 410, 510, 610 described herein is shown and comprises a flexible elongated proximal segment 770a and a distal segment 770b bonded to, or otherwise affixed to, a distal end of the proximal segment 770a. This differs from the tubular members 11, 111, 211, 311, 411, 511, 611 according to the microcatheters 10, 110, 210, 310, 410, 510, 610 of the various embodiments hereof, respectively, in that, in the various embodiments, the tubular member 11, 111, 211, 311, 411, 511, 611 is shown constructed as a one-piece, flexible elongated body formed from a single material that extends from a proximal end thereof continuously to a distal end thereof. The tubular member 711 according to the present variation is a two-piece construction in which the material selected for the proximal segment 770a thereof is different from the material selected for the distal segment 770b thereof. In this manner, proximal segment 770a may be constructed from a highly flexible, preferably polymeric, material that permits easy navigation through a patient's vasculature in order to position a distal end 711b thereof proximate an occlusion, whereas distal segment 770b may be constructed from a stiffer, more elastic material that enhances the ability of the perforations 720a, 720b to capture the occlusion as described in more detail above.

For example, proximal segment 770a of the tubular member 711 may be constructed from a flexible polymeric material that provides optimum navigability of the microcatheter through the patient's vasculature from the point of entry thereof, for instance, the patient's ulnar or radial artery, to the point of treatment, for instance, an artery in the patient's brain, a distance that can sometimes be greater than 45-100 cm. Navigating the tortuous path between the point of entry and the point of treatment requires the microcatheter, and more particularly, the tubular member 711 thereof, to be highly flexible, with sufficient column stiffness to provide effective pushability, while at the same time having kink-resistance. This is even more necessary when a clinician chooses, for whatever reason, to use the femoral or iliac artery as the point of entry, in which case, the path therefrom through the patient's vasculature to the point of treatment will be much more tortuous and involve distances that can be as much as 90-200 cm.

But choosing a flexible material oftentimes comes at the cost of stiffness, a material property that enhances the ability of the shape-changing perforations described herein to grasp onto an occlusion, or fragments thereof, as described above. As such, distal segment 770b of the tubular member 711 according to the present variation of the embodiments hereof may be constructed from a stiffer material, which in some instances can be a metallic or metallic alloy material such as nitinol, stainless steel or titanium. However, such stiffer materials are not ideally suited to provide the flexibility necessary to navigate a patient's vasculature, for example, in the event the entire tubular member were constructed from, for instance, nitinol. A two-piece tubular member 711 according to the present variation of the embodiments hereof is optimized to enhance the occlusion-grasping function of the shape-changing perforations while at the same time to provide sufficient flexibility to permit navigating the patient's vasculature. Ideally, the distal segment 770b of the tubular member 711 has a length that is only as long as is necessary to provide a sufficient number of perforations, arranged as described elsewhere herein, and may, for example, be just long enough to fit between inflatable members 230a, 230b as shown, for instance, in FIG. 3. For example, distal segment 770b may be 3-5 cm long.

Conventional manufacturing methods and techniques known to those of ordinary skill in the art may be used to bond the proximal and distal segments 770a, 770b, respectively, to one another to form a continuous interior and exterior surface. In those embodiments where tubular members provide a lumen, for example, to apply either a source of positive pressure or a source of vacuum, distal segment 770b may also include a lumen, extending either partially or completely therethrough, so as to provide a continuous path of fluid communication from the lumen of the proximal segment 770a to the lumen of the distal segment 770b.

As an alternative embodiment to the embodiment shown and described in FIGS. 16-21 hereof, the distal end 661b of the inner tubular member 661 may include an opening (not shown) through which a microguidewire (not shown) might pass from within the lumen 662 for the purpose of facilitating placement of the microcatheter 610 in the patient's vasculature.

Referring to FIG. 22, a microcatheter 810 according to still another embodiment of the present invention is shown having several components in common with the embodiments hereof previously described. Unlike the previous embodiments, though, the microcatheter 810 of the present embodiment comprises an outer tubular member 851 and an inner tubular member 861 slidably and rotatably positionable within a lumen 852 of the outer tubular member 851. Inner tubular member 861 includes a lumen 862 therein extending along the longitudinal axis from a proximal end 861a to an open distal end 861b. Inner tubular member lumen 862 is sized to permit a standard microguidewire (not shown) to move therein such as, for instance, using the microguidewire to position the microcatheter 810 as will be described in greater detail below.

Outer tubular member 851 includes an open proximal end 851a and an open distal end 851b defining an opening through which distal end 861b of the inner tubular member 861 may pass distally from within the lumen 852. Inner tubular member 861 includes an outer diameter that is smaller than an inner diameter of outer tubular member 851, thereby defining an annular interstitial space 880 (FIG. 25) extending along the longitudinal axis LA between the inner and outer tubular members 861, 851, respectively. A source of suction (not shown) may be provided, such as by vacuum or negative pressure system, in communication with the perforations 820a, 820b via the interstitial space 880 to capture grasp, grip, hold or retain an occlusion OC, or the fragments thereof, against the sidewall 863 of the outer tubular member 851 of the microcatheter 810 to more fully and completely remove the occlusion OC from within the patient's vasculature. Alternatively, a source of treatment solution (not shown) may be provided under nominal pressure in communication with the perforations 820a, 820b via the interstitial space 880 to deliver treatment solution to the treatment region for the purpose of, for example, dissolving or partially dissolving an occlusion OC.

Similar to certain other embodiments described herein, for example, the microcatheter 610 shown in FIGS. 16-20, microcatheter 810 according to the present embodiment may include one or more inflatable members 830a, 830b, such as inflatable balloons, connected, either individually or together, to a source of low pressure (not shown), such as, for example, a pre-filled syringe containing either air or saline, in fluid communication with inflatable members 830a, 830b by, for example, a lumen (not shown) provided in the sidewalls of the outer and inner tubular members 851, 861, respectively, extending longitudinally along, and spaced radially from, longitudinal axis LA. Alternatively, the source of low pressure (not shown) may be in fluid communication with inflatable members 830a, 830b by one or more separate tubular members (not shown) running longitudinally alongside an outer surface of either the outer or inner tubular members 851, 861, respectively.

According to the present embodiment, inflatable members 830a, 830b include a proximal inflatable member 830a affixed to the outer tubular member 851 spaced proximally along the longitudinal axis LA from the distal end 851b thereof a distance such that it is located proximal of a perforated section defined by the perforations 820a, 820b. One of ordinary skill in the art will appreciate that the proximal inflatable member 830a is expandable in a radial direction relative to the outer tubular member 851. Inflatable members 830a, 830b also include a distal inflatable member 830b affixed to the inner tubular member 861 near the distal end 861b thereof. One of ordinary skill in the art will also appreciate that the distal inflatable member 830b is also expandable in a radial direction relative to the inner tubular member 861.

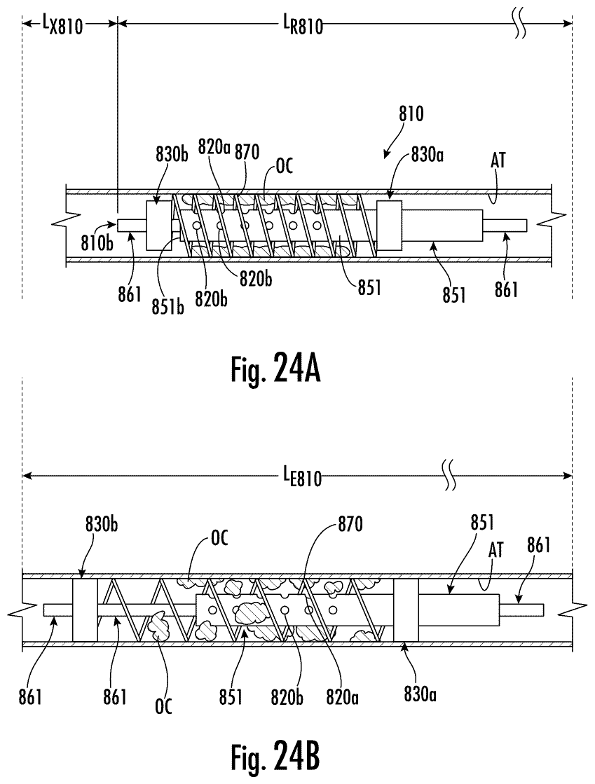

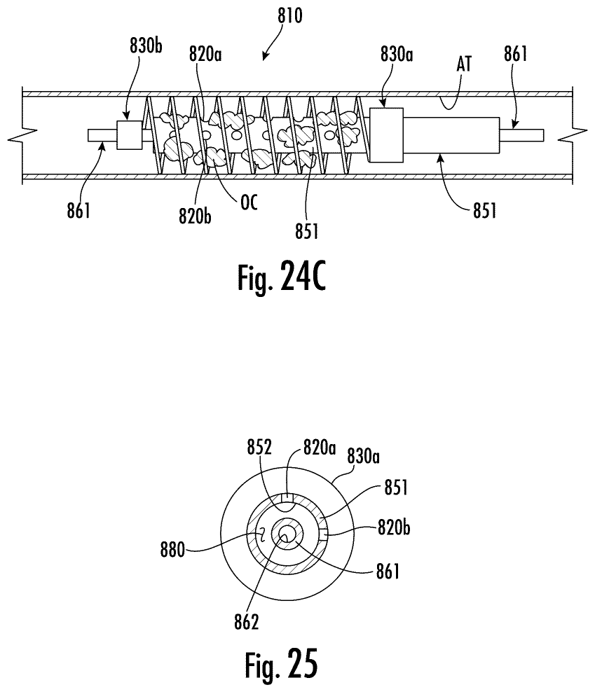

The microcatheter 810 of the present embodiment shows the inflatable members 830a, 830b in a collapsed, deflated state (FIGS. 24A, 24C) and an expanded, inflated state (FIG. 24B). One of ordinary skill in the art will understand, upon reading the present disclosure, and in particular, as the present disclosure relates to the various other embodiments hereof, that inflatable members 830a, 830b may be deflated, individually or simultaneously, to reach a deflated state (not shown). Moreover, distal inflatable member 830b may include a profile that permits it to be withdrawn into the open distal end 851b of the outer tubular member 851, and more particularly, to be withdrawn into the lumen 852 that defines the open distal end 851b of the outer tubular member 851, when the distal inflatable member 830b is in the deflated state.

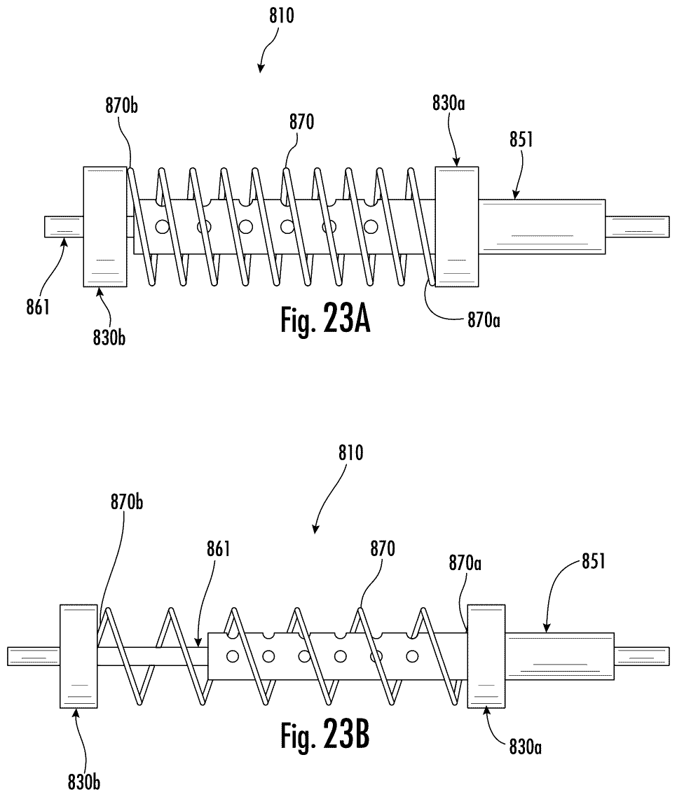

With reference now also to FIGS. 23A and 23B, the microcatheter 810 according to the present invention is shown with an optional extensible member 870 affixed at a proximal end 870a thereof to the outer tubular member 851 near the proximal inflatable member 830a, and affixed at a distal end 870b thereof to the inner tubular member 861 near the distal inflatable member 830b. According to one variation of the present embodiment, extensible member 870 is shown as a compression spring-like coil structure with multiple loops, winds or coils wrapped around tubular members 851, 861 along the longitudinal axis LA of the microcatheter 810 between the proximal and distal inflatable members 830a, 830b, respectively. Extensible member 870 is configured to have a compressed state, as shown in FIG. 23A, when the inner tubular member 861 is withdrawn into the outer tubular member 851, and configured to have an extended state, as shown in FIG. 23B, when the inner tubular member 861 is extended distally relative to the outer tubular member 851. The number of coils and the physical properties of the materials chosen therefor can be selected in any manner to optimize the function of the extensible member 870 as more fully described herein. The configuration of the extensible member 870 shown and described herein is only to illustrate the function thereof.

Unlike the extensible member 670 described in reference to the embodiment of the present invention shown in FIGS. 16-29, the proximal inflatable member 830a of the apparatus according to the preset embodiment is located proximal to the distal end 851b of the outer tubular member 851 by a larger distance so as to provide a distal length of the outer tubular member 851 over which a perforated region may be defined by perforations 820a, 820b. Extensible member 870, then, is shown to pass over this perforated region even when the inner tubular member 861 is withdrawn into the outer tubular member 851 as shown in FIG. 23A. This allows the microcatheter 810 according to the present embodiment to supply a negative vacuum force to a space radially exterior to the perforated section, one purpose of which will be described in greater detail below, even when the microcatheter 810 is in this “relaxed” state.

As shown in FIG. 23A, when the extensible member 870 is in the compressed state, individual coils thereof are spaced such that they are separated from one another by only a small distance, or alternatively, they may be touching one another. Also with reference to both FIG. 23B and FIG. 24B, extensible member 870 is configured such that when it is in the extended state, individual coils thereof are separated from one another by a distance suitable to enmesh an occlusion OC, or portions thereof, when the microcatheter 810 is advanced through a patient's vasculature such that, when the inner tubular member 861 is advanced distally relative to the outer tubular member 851, the perforations 820a, 820b are positioned radially adjacent alongside the occlusion OC and the individual coils of the extensible member 870 are spaced a greater distance longitudinally from one another. Engagement of the individual coils of the extensible member 870 into the occlusion OC, or portions thereof, can be enhanced by applying a negative pressure to the perforations 820a, 820b via lumen 862 and the interstitial space 880 (FIG. 25) such that the occlusion OC is drawn toward the surface of the outer tubular member 851 by the negative pressure, thereby entrapping the occlusion OC, or fragments thereof, deep into the individual coils of the extensible member 870. At this point, the inner tubular member 861 can be manually advanced and retracted small distances repeatedly relative to the outer tubular member 851 so as to manipulate the individual coils of the extensible member 870 longitudinally, scraping the occlusion OC (and possible the inner wall of the artery AT) and dig them even deeper into the occlusion OC, thereby capturing, grasping, gripping, holding, retaining, entrapping and enmeshing the coils firmly into the occlusion OC.

Again, by referring to the ability of the extensible member 870 to “dig into” the occlusion OC, what is meant is that, according to one embodiment of the present invention, the occlusion OC, or portions thereof, may be drawn into the space between successive coils of the extensible member 870, either under the influence of a negative pressure applied to the perforations 820a, 820b or naturally as a consequence of the coils of the extensible member 870 simply being physically close to, and embedded within, the occlusion OC, such that one or more of the individual coils of the extensible member 870 is enmeshed within the occlusion OC. Alternatively, expanding the extensible member 870 might result in the individual coils thereof being advanced into the tissue of the occlusion OC, or portions thereof.

With reference now to FIGS. 24A-24C, a method of using the microcatheter 810 according to one embodiment of the present invention will be described. The extensible microcatheter 810 according to the present embodiment of the present invention is sized and configured to be introduced into a patient's vasculature which includes the arteries of a patient's brain, the arteries surrounding a patient's heart, and any branch artery stemming therefrom. Generally, the microcatheter 810 is flexible and may assume a straight, relaxed configuration, as shown in FIG. 22, in which microcatheter 810 extends along a longitudinal axis LA from a proximal end 810a (FIG. 22) to a distal end 810b, the proximal end 810a and the distal end 810b defining a “relaxed” length L_{R810} therebetween. Before the procedure begins, the microcatheter 810 is provided in its relaxed state in which, among other things, inner tubular member 861 is withdrawn into outer tubular member 851 such that the distal inflatable member 830b (which is located on the inner tubular member 861 toward the distal end 861b thereof) is positioned near the distal end 851b of the outer tubular member 851. In addition, when the microcatheter 810 is in this relaxed state, the extensible member 870 (extending between the proximal inflatable member 830a and the distal inflatable member 830b, both of which have been deflated so as to provide a low profile) is in its “compressed” state.

Conventional devices and methods for introducing the microcatheter 810 into the patient's vasculature are utilized, including using needles, introducer sheaths, etc. (not shown) to provide percutaneous access into, for instance, the patient's radial, ulnar or femoral artery, and from there, to the rest of the patient's vasculature. Furthermore, conventional devices, such as, for example, guidewires, steering catheters, etc. (not shown) are utilized to “steer” the microcatheter 810 through the patient's vasculature toward a preselected location therein, such as, for example, the site of a vascular occlusion, blockage, obstruction, or the like, present in, for example, a cerebral artery AT of the patients' brain.

According to one method for treating a patient's vasculature using a microcatheter 810 according to the present embodiment, access is first made into the patient's vasculature, such as, for example, by a needle, dilator and/or introducer sheath (not shown). A conventional guidewire (also not shown) is inserted into the patient's vasculature via the introducer sheath and “steered” through the patient's vasculature using conventional devices and techniques until a distal tip (not shown) of the guidewire “meets” the occlusion OC. If possible, the guidewire is then advanced, under force, through the occlusion OC until it arrives beyond the distal end thereof. Thereafter, the microcatheter 810 is fed over the guidewire by inserting a proximal tip (not shown) of the guidewire into the open distal end 861b of the inner tubular member 861 then into, and through, the lumen 862 thereof. The microcatheter 810 is then advanced along the guidewire until the distal end 861b of the inner tubular member 861 “meets” a proximal end of the occlusion OC.

Once the microcatheter 810 has been inserted into and advanced through the patient's vasculature so that the distal end 861b of the inner tubular member 861 arrives to the occlusion OC, the proximal end will positioned outside the patient's body, whereas the distal end 810b is positioned within the patient's vasculature such that the distal end 810b is located proximate the occlusion OC for treatment and/or removal. While distal end 810b is shown as having a blunt tip, distal end 810b may be shaped in the form of a tapered cone, rounded front end or other, similar, non-blunt tip so as to facilitate better navigability through the patient's vasculature while minimizing trauma to tissue, etc. While the inflatable members 830a, 830b remain deflated, the microcatheter 810 is then forced through the occlusion OC until the perforations 820a, 820b are radially adjacent alongside the occlusion OC. FIG. 24A shows the inflatable members 830a, 830b in a deflated state.

Once the microcatheter 810 has been positioned in the patient's vasculature such that the perforations 820a, 820b are radially adjacent alongside the occlusion OC, the inner tubular member 861 is advanced distally an extension length L_{X810} to allow the coil structure of the extensible member 870 to open up, and the distal inflatable member 830a is inflated along with the proximal inflatable member 830a, thereby isolating the occlusion OC between the inflatable members 830a, 830b and creating a substantially sealed-in region within the artery AT surrounding the occlusion OC. Referring now also specifically to FIG. 24B, the motion of the inner tubular member 861, and more particularly, the extension of the extensible member 870, the distal end of which is affixed to the inner tubular member 861, will cause the individual coils of the extensible member 870 to physically interrupt, cut through, slice, or otherwise separate, fragments of the occlusion OC from the main body thereof. Such fragments, however, will be trapped in the region proximate the perforations 820a, 820b due to the inflated inflatable members 830a, 830b.

At this point, the source of a negative pressure is activated to draw the fragments of the occlusion OC via the interstitial space 880 and the perforations 820a, 820b toward the outer surface of the outer tubular member 851. While the source of negative pressure remains activated, either inner tubular member 861 or outer tubular member 851 may be manipulated to move distally and proximally short distances, back-and-forth, relative to the other, to cause the individual coils of the extensible member 870 to extend and collapse, thereby physically agitating the occlusion OC to further detach additional fragments of the occlusion OC from the main body thereof. As these fragments are set loose from the main body of the occlusion OC, they are drawn to the surface of the outer tubular member 851, some of which may adhere to the surface and some of which may aspirate from the patient's body entirely through the perforations 820a, 820b and the interstitial space 880. Because the inner tubular member 861 has an outer diameter that is smaller than the inner diameter of the lumen 852 of the outer tubular member 851, the open distal end 851b of the outer tubular member 851 (through which inner tubular member 861 extends) provides a distal opening (not shown) of the interstitial space 880 which can also aspirate fragments of the occlusion OC from within the interior region.

Referring now also to FIG. 24C, the microcatheter 810 is returned to the relaxed state by deflating proximal and distal inflation members 830a, 830b, respectively, and withdrawing the inner tubular member 861 toward, and at least partially within, the lumen 852 of the outer tubular member 851. This collapses the individual coils of the extensible member 870 (between which fragments of the occlusion OC have become entrapped), thereby physically grasping the fragments and increasing the efficacy of physically withdrawing any fragments left that haven't already been aspirated under the influence of the source of negative pressure applied to the perforations 820a, 820b and to the interstitial space 880. At this point, the source of negative pressure can be deactivated, although it may be maintained through the rest of the procedure to further enhance the efficacy of withdrawing the occlusion OC from within the patient's body. The microcatheter 810 is withdrawn from the patient's body, taking the occlusion OC with it.

Extensible member 870 is chosen such that it has a coil diameter that roughly approximates the lumen diameter of the artery AT. Given the general flexible nature of the material chosen for the extensible member 870, the individual coils will deform, as needed, for the microcatheter to traverse the patient's vasculature and be forced through the occlusion OC. As the microcatheter 810 is forced through the occlusion OC, though, the general nature of the coils of the extensible member will have a tendency to begin to break up the occlusion OC into fragments. Manual manipulation of the microcatheter 810 while it is being advanced to cross the occlusion, such as, for example, by moving the microcatheter 810 back-and-forth, distal then proximal, repeatedly short distances might result in increased amounts of fragments being dislodged from the occlusion OC. Generally, it is undesirable to dislodge portions of an occlusion prior to arresting blood flow in the affected artery so as not to cause fragments of the occlusion to flow freely into the patient's bloodstream, potentially causing life threatening emboli in other parts of the patient's body.

For this reason, one variation of the method for performing a treatment using the microcatheter 810 according to the present embodiment includes advancing the microcatheter 810 over the guidewire until the distal end 861 of the inner tubular member 861 meets the occlusion OC, but first advancing only the inner tubular member 861 through the occlusion OC before advancing the outer tubular member 851 through the occlusion OC so as not to prematurely agitate the occlusion OC before distal inflatable member 830b has been inflated distal of the occlusion OC, thereby arresting blood flow in the artery.

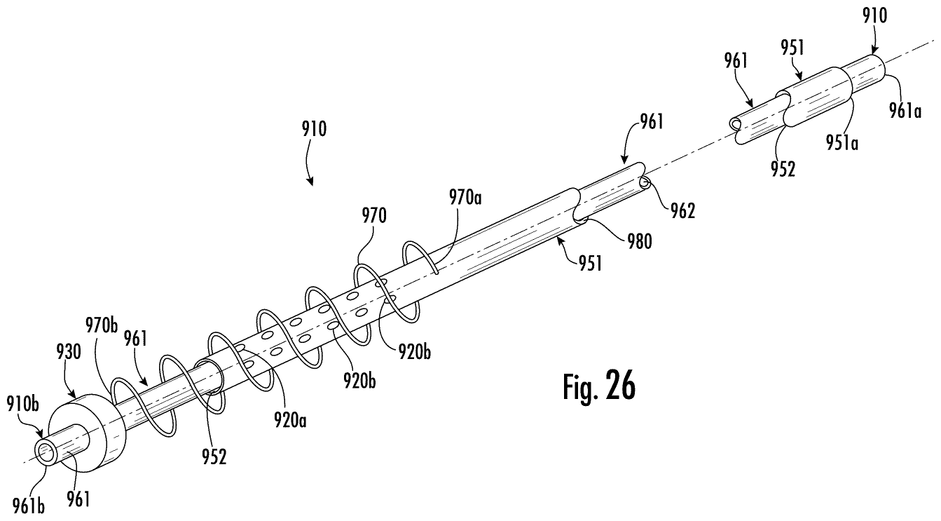

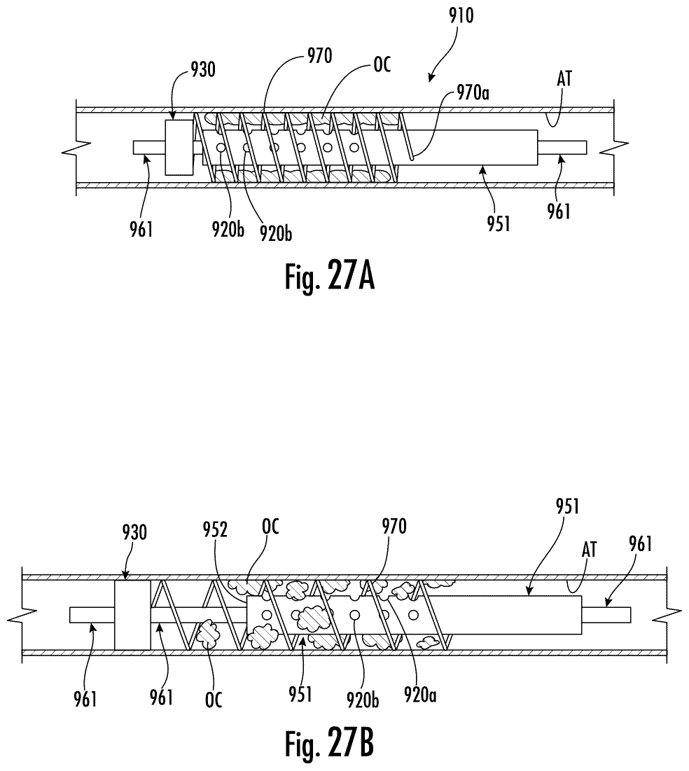

With reference to FIGS. 26-27B, a microcatheter 910 according to another embodiment of the present invention is shown having several components in common with the multiple embodiments hereof previously described, and especially with microcatheter 810 of the previous embodiment. Unlike the microcatheter 810 of the previous embodiment, however, the microcatheter 910 of the present embodiment comprises only a single inflatable member 930 located near a distal end 961b of an inner tubular member 961, which is itself slidably and rotatably positionable within a lumen 952 of an outer tubular member 951. Inner tubular member 961 includes a lumen 962 therein extending along a longitudinal axis LA from a proximal end 961a to an open distal end 961b. Inner tubular member lumen 962 is sized to permit a standard microguidewire (not shown) to move therein such as, for instance, using the microguidewire to position the microcatheter 910 as will be described in greater detail below.

Outer tubular member 951 includes an open proximal end 951a and an open distal end 951b defining an opening through which distal end 961b of the inner tubular member 961 may pass distally from within the lumen 952. Inner tubular member 961 includes an outer diameter that is smaller than an inner diameter of outer tubular member 951, thereby defining an annular interstitial space 980 extending along the longitudinal axis LA between the inner and outer tubular members 961, 951, respectively. A source of suction (not shown) may be provided, such as by vacuum or negative pressure system, in communication with the perforations 920a, 920b via the interstitial space 980 to capture grasp, grip, hold or retain an occlusion OC, or the fragments thereof, against the sidewall 963 of the outer tubular member 951 of the microcatheter 910 to more fully and completely remove the occlusion OC from within the patient's vasculature. Alternatively, a source of treatment solution (not shown) may be provided under nominal pressure in communication with the perforations 920a, 920b via the interstitial space 980 to deliver treatment solution to the treatment region for the purpose of, for example, dissolving or partially dissolving an occlusion OC.

Similar to certain other embodiments described herein, for example, the microcatheter 810 shown in FIGS. 22-25, the inflatable member 930 of microcatheter 910 may be inflated by, for example, a pre-filled syringe containing either air or saline, that is in fluid communication with the inflatable member 930 by, for example, a lumen (not shown) provided in the sidewalls of the inner tubular members 961 extending longitudinally along, and spaced radially from, longitudinal axis LA. Alternatively, the source of low pressure (not shown) may be in fluid communication with the inflatable member 930 by one or more separate tubular members (not shown) running longitudinally alongside an outer surface of the inner tubular member 961.

The microcatheter 910 of the present embodiment shows the inflatable member 930 in a collapsed, deflated state (FIG. 27A) and in an expanded, inflated state (FIG. 27B). Inflatable member 930 may have a profile that permits it to be withdrawn into the open distal end 951b of the outer tubular member 951, and more particularly, to be withdrawn into the lumen 952 that defines the open distal end 951b of the outer tubular member 951, when the inflatable member 930 is in the deflated state.

With reference now also to FIGS. 27A and 27B, the microcatheter 910 according to the present invention is shown with an optional extensible member 970 affixed at a proximal end 970a thereof to the outer tubular member 951 proximal of the perforated region, and affixed at a distal end 970b thereof to the inner tubular member 961 near the inflatable member 930. According to one variation of the present embodiment, extensible member 870 is shown as a compression spring-like coil structure with multiple loops, winds or coils wrapped around tubular members 951, 961 along the longitudinal axis LA of the microcatheter 910 extending, generally, over the perforated region. Extensible member 970 is configured to have a compressed state, as shown in FIG. 27A, when the inner tubular member 961 is withdrawn into the outer tubular member 951, and configured to have an extended state, as shown in FIG. 27B, when the inner tubular member 961 is extended distally relative to the outer tubular member 951. The number of coils and the physical properties of the materials chosen therefor can be selected in any manner to optimize the function of the extensible member 970 as more fully described herein. The configuration of the extensible member 970 shown and described herein is only to illustrate the function thereof.

As shown in FIG. 27A, when the extensible member 970 is in the compressed state, individual coils thereof are spaced such that they are separated from one another by only a small distance, or alternatively, they may be touching one another. With reference also to FIG. 27B, extensible member 970 is configured such that when it is in the extended state, individual coils thereof are separated from one another by a distance suitable to enmesh an occlusion OC, or portions thereof, when the microcatheter 910 is advanced through a patient's vasculature such that the perforations 920a, 920b are positioned radially adjacent alongside the occlusion OC. Engagement of the individual coils of the extensible member 970 into the occlusion OC, or portions thereof, can be enhanced by applying a negative pressure to the perforations 920a, 920b via lumen 962 and the interstitial space 980 such that the occlusion OC is drawn toward the surface of the outer tubular member 951 by the negative pressure, thereby entrapping the occlusion OC, or fragments thereof, deep into the individual coils of the extensible member 970. At this point, the outer tubular member 951 can be manually advanced and retracted small distances repeatedly relative to the inner tubular member 961 so as to manipulate the individual coils of the extensible member 970 longitudinally, scraping the occlusion OC (and possible the inner wall of the artery AT) and dig them even deeper into the occlusion OC, thereby capturing, grasping, gripping, holding, retaining, entrapping and enmeshing the coils firmly into the occlusion OC.

Again, by referring to the ability of the extensible member 970 to “dig into” the occlusion OC, what is meant is that, according to one embodiment of the present invention, the occlusion OC, or portions thereof, may be drawn into the space between successive coils of the extensible member 970, either under the influence of a negative pressure applied to the perforations 920a, 920b or naturally as a consequence of one or more of the coils of the extensible member 970 simply being physically enmeshed within the occlusion OC. Alternatively, expanding the extensible member 970 might result in the individual coils thereof being advanced into the tissue of the occlusion OC, or portions thereof.

With continued reference to FIGS. 27A-27B, a method of using the microcatheter 910 according to one embodiment of the present invention will be described. The extensible microcatheter 910 according to the present embodiment of the present invention is sized and configured to be introduced into a patient's vasculature which includes the arteries of a patient's brain, the arteries surrounding a patient's heart, and any branch artery stemming therefrom. Generally, the microcatheter 910 is flexible and may assume a straight, relaxed configuration, as shown in FIG. 26, in which microcatheter 910 extends along a longitudinal axis LA from a proximal end 910a (FIG. 26) to a distal end 910b, the proximal end 910a and the distal end 910b defining a “relaxed” length L_{R910} therebetween. Before the procedure begins, the microcatheter 910 is provided in its relaxed state in which, among other things, inner tubular member 961 is withdrawn into outer tubular member 951 such that the inflatable member 930 (which is located on the inner tubular member 961 near the distal end 961b thereof) is positioned near the distal end 951b of the outer tubular member 951. In addition, when the microcatheter 910 is in this relaxed state, the extensible member 970 is in its “compressed” state and the inflatable member 930 is in a deflated, low profile state.

The extensible member according to this embodiment, or to any of the embodiments of the present invention in which a coil-like extensible member is described, may be adapted to provide a “supercoiled” compressed configuration in which the extensible member provides superior physical engagement, disruption and capture with an occlusion. According to this adaptation, the outer tubular member (to which the proximal end of the extensible member is affixed) is advanced distally while the inner tubular member (to which the distal end of the extensible member is affixed) is held in position, thereby compressing the coil-like extensible member along its extension along its helix axis until each of the individual coils abuts its neighboring coil, forming an approximated tube-like structure. With force, the outer tubular member can be further advanced distally, causing the outermost coil to be forced within the tube-like structure, effectively inverting the proximal end of the extensible member, and forcing the next-most distal coil to be urged radially outwardly, thereby increasing the diameter of the extensible member. Further distal advancement of the proximal end of the extensible member, then, causes additional subsequent coils to be bunched together and stacked further outwardly, not only increasing the overall diameter of the extensible member at this axial location, but also increasing the “mass” and stiffness thereof due to the bunching of successive individual coils that are stacked on top of one another. The cross-sectional profile of the coil can be selected to enhance the ability of the extensible member to achieve this “supoercoiled” configuration.

Conventional devices and methods for introducing the microcatheter 910 into the patient's vasculature are utilized, including using needles, introducer sheaths, etc. (not shown) to provide percutaneous access into, for instance, the patient's radial, ulnar or femoral artery, and from there, to the rest of the patient's vasculature. Furthermore, conventional devices, such as, for example, guidewires, steering catheters, etc. (not shown) are utilized to “steer” the microcatheter 910 through the patient's vasculature toward a preselected location therein, such as, for example, the site of a vascular occlusion, blockage, obstruction, or the like, present in, for example, a cerebral artery AT of the patients' brain.

According to one method for treating a patient's vasculature using a microcatheter 910 according to the present embodiment, access is first made into the patient's vasculature, such as, for example, by a needle, dilator and/or introducer sheath (not shown). A conventional guidewire (also not shown) is inserted into the patient's vasculature via the introducer sheath and “steered” through the patient's vasculature using conventional devices and techniques until a distal tip (not shown) of the guidewire “meets” the occlusion OC. If possible, the guidewire is then advanced, under force, through the occlusion OC until it arrives beyond the distal end thereof. Thereafter, the microcatheter 910 is fed over the guidewire by inserting a proximal tip (not shown) of the guidewire into the open distal end 961b of the inner tubular member 961 then into, and through, the lumen 962 thereof. The microcatheter 910 is then advanced along the guidewire until the distal end 961b of the inner tubular member 961 “meets” a proximal end of the occlusion OC.

Once the microcatheter 910 has been inserted into and advanced through the patient's vasculature so that the distal end 961b of the inner tubular member 961 arrives to the occlusion OC, the proximal end will positioned outside the patient's body, whereas the distal end 910b is positioned within the patient's vasculature such that the distal end 910b is located proximate the occlusion OC for treatment and/or removal. While distal end 910b is shown as having a blunt tip, distal end 910b may be shaped in the form of a tapered cone, rounded front end or other, similar, non-blunt tip so as to facilitate better navigability through the patient's vasculature while minimizing trauma to tissue, etc. While the inflatable member 930 remains deflated, the microcatheter 910 is then forced through the occlusion OC until the inflatable member 930 arrives to a position distal of the occlusion OC and the perforations 920a, 920b are radially adjacent alongside the occlusion OC. Alternatively, the microcatheter 910 may be advanced further such that the perforations 920a, 920b are located just slightly distal of the occlusion OC.

Inflatable member 930 is now inflated, thereby expanding radially until it meets with the inner surface of the artery AT, thereby acting as an “anchor” for reasons described next. Outer tubular member 951 is then withdrawn proximally by an extension length L_{X810} while inner tubular member 961 remains in a fixed position axially, thereby allowing the coil structure of the extensible member 970 to open up, physically interrupting, cutting through, slicing, or otherwise separating, fragments of the occlusion OC from the main body thereof. Such fragments, however, will be prevented from travelling distally to other regions of the patient's body due to the inflated inflatable member 930.

At this point, the source of a negative pressure is activated to draw the fragments of the occlusion OC via the interstitial space 980 and the perforations 920a, 920b toward the outer surface of the outer tubular member 951. While the source of negative pressure remains activated, the outer tubular member 851 may be manipulated to move distally and proximally short distances, back-and-forth, relative to the other to cause the individual coils of the extensible member 970 to extend and collapse, thereby physically agitating the occlusion OC to further detach additional fragments of the occlusion OC from the main body thereof. As these fragments are set loose from the main body of the occlusion OC, they are drawn to the surface of the outer tubular member 951, some of which may adhere to the surface and some of which may aspirate from the patient's body entirely through perforations 920a, 920b and the interstitial space 980.

To remove the microcatheter 910 from the patient's body along with the occlusion OC, or fragments thereof, the microcatheter is first returned to the relaxed state by advancing the outer tubular member 951 relative to the inner tubular member 961 (which is held in place by the inflated inflatable member 930). This collapses the individual coils of the extensible member 970 (between which fragments of the occlusion OC have become entrapped), thereby physically grasping the fragments and increasing the efficacy of physically withdrawing any fragments left that haven't already been aspirated under the influence of the source of negative pressure applied to the perforations 920a, 920b and to the interstitial space 980. Inflatable member 930 may be inflated entirely, although it may be preferable to only partially deflate the inflatable member 930 so that it can gently slide across the inner surface of the artery AT as the microcatheter 910 is being removed, thereby bringing along with any residual fragments of the occlusion OC with remain adhered to the inner surface of the artery AT. At this point, the source of negative pressure can be deactivated, although it may be maintained through the rest of the procedure to further enhance the efficacy of withdrawing the occlusion OC from within the patient's body. The microcatheter 910 is withdrawn from the patient's body, taking the occlusion OC with it.

One of ordinary skill in the art will appreciate, upon reading the within disclosure, that although the apparatuses according to the various embodiments hereof have been described with reference, preferably, to treating an occlusion present within one of the arteries of a patient's brain, the apparatuses may be adapted to treat occlusions present within the other arteries or organs of a patient's body, for instance, a pulmonary embolism present within an artery of a patient's lung, or a DVT present in a lower extremity of a patient's limb.

One of ordinary skill in the art will also appreciate, upon reading the within disclosure, that while the present invention has been described with reference to apparatuses in which a portion or feature thereof, such as, for example, inner tubular members 211, 311, 411, 711 and inner tubular members 661, 861, 961, are advanced “through” an occlusion (as shown, for example, in which such feature passes through the middle region of the occlusion such that the feature is surrounded by the occlusion once in position), any of the apparatuses according to the various embodiments of the present invention can be used for the purposes described herein even if the apparatus is navigated “around” or to one side or the other of the occlusion. This is to say that each patient's physiology is unique and the manner in which any patient presents his/her unique physiology might include an equally-unique size, shape, orientation and composition of an occlusion such that when an apparatus according to any embodiment of the present invention is used as described herein, such apparatus might not, in fact, penetrate the occlusion by traversing directly through its center. Rather, it might cross the occlusion on the side, etc.

While exemplary methods and compositions have been illustrated by describing examples, and while the examples have been described in considerable detail, it is not the intention of the applicant to restrict or in any way limit the scope of the appended claims to such detail. It is, of course, not possible to describe every conceivable combination of components or methodologies for purposes of describing the systems, methods, devices, and so on, described herein. Additional advantages and modifications will readily appear to those skilled in the art. Therefore, the invention in not limited to the specific details, the representative revascularization catheter system, and the illustrative examples shown and described. Thus, this application is intended to embrace alterations, modifications, and variations that fall within the scope of the appended claims. Furthermore, the preceding description is not meant to limit the scope of the invention. Rather, the scope of the invention is to be determined by the appended claims and their equivalents.

What is claimed is:

1. A microcatheter system for removing an occlusion from a patient's vasculature, comprising:
an outer tubular member having an outer surface, a proximal end, a distal end, and a central lumen extending from the proximal end toward the distal end, wherein the central lumen and the outer surface cooperate to define a side wall radially therebetween;
one or more perforations formed in the side wall of the outer tubular member near the distal end thereof, the one or more perforations being in fluid communication with the central lumen of the outer tubular member;
an inner tubular member slidably positioned within the central lumen of the outer tubular member, the inner tubular member having a distal end extending distally from a distal opening in the outer tubular member; and,
an extensible member having a proximal end affixed to the outer tubular member and a distal end affixed to the inner tubular member, wherein the inner tubular member is advanceable distally relative to the outer tubular member to expand the extensible member.

2. The microcatheter system of claim 1, further comprising:
at least one inflatable member affixed to the inner tubular member near the distal end thereof.

3. The microcatheter system of claim 2, wherein the at least one inflatable member comprises a proximal inflatable member affixed to the inner tubular member and a distal inflatable member affixed to the outer tubular member and wherein the at least one or more perforations are disposed therebetween.

4. The microcatheter system of claim 1, wherein at least one of the one or more perforations comprises one or more perforations having a first shape and one or more perforations having a second shape.

5. The microcatheter system of claim 4, wherein the first shape of the one or more perforations is a slit.

6. The microcatheter system of claim 4, wherein the second shape of the one or more perforations is a substantially circular shape.

7. The microcatheter system of claim 1, further comprising:
a source of suction in fluid communication with the central lumen of the outer tubular member near the proximal end thereof.

8. The microcatheter system of claim 1, wherein the central lumen is open at both the proximal and distal ends of the outer tubular member.

9. The microcatheter system of claim 1, wherein when the outer tubular member is in the relaxed state, at least one of the one or more of the one or more perforations are in a relaxed configuration.

10. The microcatheter system of claim 1, wherein when the outer tubular member is in the extended state, at least one of the one or more of the one or more perforations are in an open configuration.

11. The microcatheter system of claim 1, wherein the extensible member is a coil superimposed over at least a portion of the outer tubular member proximate the one or more perforations, wherein the extensible member is advanceable along the outer surface of the outer tubular member between a collapsed configuration in which individual loops of the coil are spaced a first distance from one another and an extended configuration in which individual loops of the coil are spaced a second distance from one another, and wherein the second distance is greater than the first distance.

12. The microcatheter system of claim 1, wherein the inner tubular member comprises one or more perforations formed in a sidewall thereof.

13. The microcatheter system of claim 12, wherein the inner tubular member comprises a central lumen, and wherein the one or more perforations are in fluid communication with the central lumen.

14. The microcatheter system of claim 1, wherein the extensible member is an expandable stent.

15. The microcatheter system of claim 14, wherein the proximal end of the expandable stent is affixed to an inner surface of the central lumen of the outer tubular member.

16. The microcatheter system of claim 1, wherein the inner tubular member is a guidewire.

17. The microcatheter system of claim 2, wherein the at least one inflatable member has a preselected profile.

18. The microcatheter system of claim 2, wherein the at least one inflatable member comprises a proximal inflatable member affixed to the outer tubular member and a distal inflatable member affixed to the outer tubular member and wherein the at least one or more perforations are disposed therebetween.

19. The microcatheter system of claim 18, wherein the extensible member extends between the proximal inflatable member and the distal inflatable member.

20. The microcatheter system of claim 1, wherein the extensible member is in spaced relation to the outer surface of the outer tubular member.
